(19) United States
(12) Patent Application Publication
(10) Pub. No.: US 2004/0228817 A1
(43) Pub. Date: Nov. 18, 2004

(54) Makeup combining at least one photochromic dye and at least one goniochromatic agent

(75) Inventors: Jean-Christophe Simon, Alsbach-Haehnlein (DE); Xavier Blin, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/687,632

(22) Filed: Oct. 20, 2003

Related U.S. Application Data
(60) Provisional application No. 60/434,408, filed on Dec. 19, 2002.
(60) Provisional application No. 60/434,406, filed on Dec. 19, 2002.

(30) Foreign Application Priority Data
Oct. 18, 2002 (FR) 02 13037
Oct. 18, 2002 (FR) 02 13035

(51) Int. Cl. A61K 7/021

(52) U.S. Cl. 424/63

(57) ABSTRACT

The present invention relates to a cosmetic Product containing at least a first composition and a second composition, the first composition containing at least one first dye in a first physiologically acceptable medium, and the second composition containing at least one second dye in a second physiologically acceptable medium, the first dye being photochromic and the second dye being at least one goniochromatic coloring agent.

### REFERENCE TO PRIOR APPLICATIONS

This application claims priority to U.S. provisional applications 60/434,406 and 60/434,408 both filed Dec. 19, 2002, and to French patent applications 0213037 and 0213035 both filed Oct. 18, 2002, all incorporated herein by reference.

### FIELD OF THE INVENTION

The present invention relates to a novel cosmetic product, especially for making up the skin, the lips and/or the integuments, combining at least one goniochromatic coloring agent and at least one photochromic dye.

The term “makeup product” means a product containing a dye for depositing a color onto a human keratin material (the skin, the lips or the integuments), such as a lipstick, a makeup rouge, an eyeliner, a foundation, a self-tanning product, a makeup product having care properties or a semi-permanent makeup product (tattoo).

More particularly, the product under consideration according to the invention comprises at least two cosmetic compositions that may be applied successively not only to the human face but also the body, the human lower and upper eyelids, the lips and the integuments, for instance the nails, the eyebrows, the eyelashes or the hair, and also a two-coat makeup process for the human face and body.

The term “cosmetic composition” denotes a composition as defined in Council Directive 93/35/EEC of 14 Jun. 1993.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

One of the main functions of makeup consists in providing optical and/or color effects to the makeup areas on the skin, the lips, the eyelashes and the nails.

In general, these effects are static, i.e. they do not change either over time or as a function of the ambient luminosity. The change most frequently encountered generally consists of a gradual alteration over time of the original optical and/or color effects. This alteration is generally due either to a transfer and/or migration of the composition, these phenomena being particularly pronounced for lipsticks, or to poor behaviour of the pigments present in the makeup composition with respect to sebum, this phenomenon more particularly being encountered with foundations.

Thus, at the present time, no makeup product exists that might be described as dynamic in terms of shade, i.e. capable of changing significantly in terms of color and/or radiance in response, for example, to a change in the ambient luminosity.

Now, it is clear that a makeup product of this type would precisely satisfy a desire of consumers who are in search of novel colored makeups contrasting with conventional makeup compositions.

One main objective of the invention is, specifically, to satisfy this need. More particularly, the invention is directed towards providing a cosmetic product that has a “dynamic” optical and/or color effect.

In the present instance, the present invention especially exploits the ability manifested by certain photochromic dyes to change color when they are subjected to a radiation containing ultraviolet rays, and to do so reversibly.

The use of photochromic pigments in cosmetic compositions is known. Patent application EP 970 689 describes a two-coat makeup product for superimposing a top coat containing an ultraviolet-screening agent over a base coat containing a photochromic pigment. The corresponding makeup makes it possible to form patterns that appear or disappear depending on the nature of the light, but no dynamic effect in terms of color is associated therewith. Patent application WO 02/078665 proposes, for its part, cosmetic compositions comprising photochromic agents of naphthopyran type. However, these agents are incorporated into the cosmetic composition in a liquid form encapsulated in a microcapsule. This dispersible form is described as having antioxidant activity.

### DETAILED DESCRIPTION OF THE PREFERRED EMBODIMENTS

The inventors have found, unexpectedly, that it is particularly advantageous to combine at least one goniochromatic dye with at least one specific photochromic dye to obtain a novel dynamic effect in terms of color and radiance.

In the present instance, according to a first aspect, the present invention relates to a cosmetic product especially for making up the skin, the lips and/or the integuments, comprising at least a first composition and a second composition, the first composition comprising at least one dye referred to as the “first dye” preferably in a physiologically acceptable medium, and the second composition comprising at least one dye referred to as the “second dye” also preferably in a physiologically acceptable medium, the first dye being photochromic and the second dye being at least one goniochromatic coloring agent, the first and second dyes being different from one another. The physiologically acceptable media of the first and second compositions may be the same or different.

According to one particular variant, the first photochromic dye is of naphthopyran type.

According to a second aspect, the present invention also relates to a cosmetic product especially for making up the skin, the lips and/or the integuments, comprising at least a first composition and a second composition, the first composition comprising at least one dye referred to as the “first dye” optionally in a physiologically acceptable medium, and the second composition comprising at least one dye referred to as the “second dye” optionally in a second physiologically acceptable medium, the first dye being of naphthopyran type and the second dye being at least one goniochromatic coloring agent.

Advantageously, the combination of the first dye in accordance with the invention, i.e. the photochromic dye and/or the dye of naphthopyran type, with at least one goniochromatic coloring agent in a cosmetic product more particularly intended for makeup makes it possible to give this cosmetic product a rapid and reversible dynamic effect in terms of a shade and/or radiance effect. Thus, the difference in shade is sufficiently significant to be noticed with the naked eye. Moreover, it is immediate, i.e. it takes place within a very short period, either after exposing the makeup to UV radiation, or after stopping this exposure.

Besides this “dynamic shade and/or radiance effect” aspect, the products according to the invention are most particularly advantageous for compensating for the color alteration phenomenon discussed previously, which is generally associated with poor behaviour of conventional pigments with respect to sebum. Specifically, combining these pigments with a first dye in accordance with the invention advantageously makes it possible to compensate for this type of alteration, since the dye is capable by itself, in response to light excitation of UV type, of yielding a colored effect equivalent to that initially afforded by the pigments.

As stated previously, the product according to the invention preferably comprises, in sum, two (or more) overall physiologically acceptable compositions.

The compositions may be packaged separately or together in the same packaging article or in two (or more) separate or distinct packaging articles. Each composition may be a free or compacted powder, a foundation, a makeup rouge, an eyeshadow, a concealer product, a blusher, a lipstick, a lip balm, a lip gloss, a lip pencil, an eye pencil, a mascara, an eyeliner, a nail varnish or a body makeup product or skin-coloring product. In the present instance, the first composition of the product according to the invention may constitute a base coat applied to the support to be made up, especially the lips, and the second composition may constitute a top coat. However, it is possible to apply, under the first coat, an undercoat possibly having the constitution of the second composition. It is also possible to deposit an overcoat onto the second coat, the constitution of which may or may not be identical to that of the first coat.

According to another of its aspects, the invention relates to a makeup kit containing a cosmetic makeup product as defined above, in which the various compositions may especially be packaged separately and are accompanied by suitable application means. These means may be small or large brushes, pens, pencils, felts, nibs, sponges and/or foams.

According to another of its aspects, the invention also relates to a process for making up the skin, the lips and/or the integuments, which consists in applying to the skin, the lips and/or the integuments a cosmetic makeup product as defined above.

More particularly, the first composition comprising at least one dye, referred to as the first dye, in a first physiologically acceptable medium is applied, as a first coat, and a coat of the second composition comprising at least one goniochromatic coloring agent, also referred to as the second dye, in a second physiologically acceptable medium is then applied, over all or part of the first coat.

In particular, the first dye is photochromic and/or of naphthopyran type.

Preferably, the makeup obtained is a two-coat makeup. In particular, the base coat is a foundation, a makeup rouge, a lipstick, a lip gloss, an eyeliner or a body makeup product, and the top coat is a protective or care product.

Preferably, the first coat of the first composition, referred to as the base coat is allowed to dry before applying the second coat of the composition.

The order of superposition and/or the mode of superposition of the two compositions, i.e. total or partial, may moreover be advantageous for imparting additional visual effects. The second coat may be arranged, for example, so as to form patterns on the surface of the base coat. Thus, when the top coat contains the photochromic dye, it is possible to apply the second coat onto only a part of the first coat containing the goniochromatic agent. Only the part onto which the two coats have been superimposed will be capable of displaying a color effect in the absence of UV radiation. In this regard the base coat and surface coat may each constitute a pattern with only partial overlap. In addition, multiple alternate layers of base and second coat may be applied.

This two-coat makeup may be adapted for any product for making up the skin not only of the human face but also of the scalp and the body, mucous membranes, for instance the lips and the inner edge of the lower eyelids, and the integuments, for instance the nails, the eyelashes, the hair, the eyebrows or even body hair.

Photochromic Dye

In general, a photochromic dye is a dye that has the property of changing shade when it is illuminated with ultraviolet light and of regaining its initial color when it is no longer illuminated with this light, or of passing from an uncolored form to a colored form and vice versa. In other words, such a dye presents different shades depending on whether it is illuminated with light containing a certain amount of UV radiation, for instance sunlight, or with artificial light.

For the purposes of the present invention, the term “photochromic dye” denotes a dye characterized by a difference in shade ΔE at least equal to 5.

For the purposes of the present invention, ΔE features the difference in shade observed for the photochromic dye between its excited state, i.e. in the presence of UV irradiation, and its unexcited state, i.e. in the absence of UV irradiation.

The ΔE may be determined from the chromatic space and more particularly from the specific chromatic coordinates of the photochromic dye under consideration, evaluated firstly after exposure to UV radiation for 2 minutes, generally using artificial light containing UV radiation, and secondly at rest, i.e. 5 seconds after stopping the radiation.

More specifically, the measurement protocol is as follows:

1% by weight of the photochromic dye under consideration is formulated in 100% by weight of a white lipstick base having the composition below:

| | |
|---|---|
| 2-octyldodecanol | 0.5% |
| hectorite modified with distearyl | 0.6% |
| dimethylammonium chloride | |
| liquid lanolin | 27.2% |
| microcrystalline wax | 10.5% |
| polyglycerolated (3 mol) beeswax | 4.2% |
| acetylated lanolin | 6.7% |
| arara oil (oleic acid esters) | 13.5% |
| oxypropylenated (5 PO) lanolin wax | 6.7% |
| oleyl erucate | 13.5% |
| oleic-linoleic-linolenic acid triglycerides | 1.7% |
| palmitic-oleic-linoleic acid triglycerides | 13.5% |
| sodium hyaluronate | 0.1% |
| preserving agents | 0.1% |
| vitamin | 0.5% |
| UV-screening agent | 0.7% |

A layer 50 μm thick of the composition is spread onto a Leneta brand contrast card of reference Form 1A Penopac, using an automatic spreader. The layer covers at least the white background of the card.

Reflection measurements are performed using a Minolta 3700D spectrocolorimeter. The initial trichromatic coordinates of the composition before irradiation (Lₒ, aₒ and bₒ) are thus determined. The composition is then subjected to a flux of 2 mW/cm² of a UVA radiation for two minutes, and the new trichromatic coordinates (L, a and b) are then determined immediately after stopping the irradiation. Less than 5 seconds elapse between stopping the irradiation and determining the new coordinates.

The ΔE is calculated in the following manner:

ΔE=[(L−Lₒ)²+(a−aₒ)²+(b−bₒ)²]^{1/2}

Advantageously, the photochromic dyes according to the invention have a ΔE value at least equal to 5, especially greater than or equal to 10, in particular greater than or equal to 25, more particularly greater than or equal to 35, or even greater than or equal to 45.

A ΔE measurement of greater than about 30 means that the photochromic dye gives a bright color.

In the context of the present invention, the photochromic dyes moreover have the advantage of responding quickly either to an excitation with UV light or to stoppage of such an excitation. This results in a rapid change in terms of coloration. This change in shade may thus be reflected in a time interval advantageously of less than or equal to 2 minutes, especially less than or equal to 1 minute and in particular less than or equal to 50 seconds.

According to one variant of the invention, the product according to the invention contains at least two photochromic dyes as defined above, especially in the same composition, optionally in combination with a non-photochromic dye.

The photochromic dye under consideration according to the invention may be present in any amount, for example an amount ranging from 0.001% to 20% by weight, especially from 0.005% to 10% by weight, in particular from 0.01% to 5% by weight and more particularly from 0.05% to 2% by weight, or even from 0.1% to 1% by weight, relative to the total weight of the composition containing it.

The photochromic dyes according to the invention may be present in any form including a dissolved or dispersed form in the first or second composition. According to one particular variant, they are present therein in a dissolved form.

More preferably, the photochromic dyes under consideration according to the invention are of organic nature.

Even more preferably, the photochromic organic dyes according to the invention are naphthopyrans and especially as defined below.

Dyes of Naphthopyran Type

The products according to the invention may comprise a first composition comprising at least one naphthopyran derivative as first dye. This derivative may be chosen more particularly from 3H-naphtho[2,1-b]pyrans, which may be represented by formula (I), or from 2H-naphtho[1,2-b]pyrans, which may be represented by formula (II):

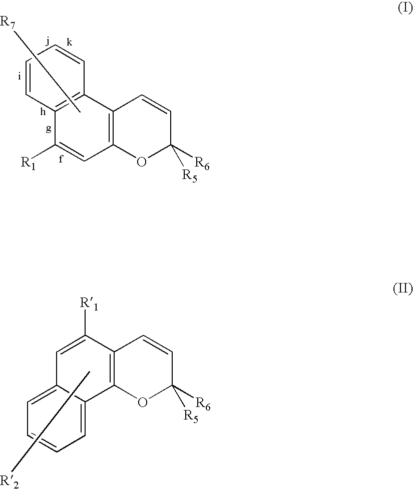

in which:

R1 represents:

(i) a hydrogen atom;

(ii) a linear, branched or cyclic, saturated or unsaturated hydrocarbon-based group containing from 1 to 30, preferably 1 to 18 and better still 1 to 12, or even 1 to 6, carbon atoms, optionally comprising 1 to 5 hetero atoms selected from the group consisting of N, O, S, Si and P, and/or optionally halogenated or perhalogenated, especially with F, Br and/or Cl;

(iii) a hydrocarbon-based ring formed with one of the bonds “f” or “gh” and the radical R₇; or

(iv) a group selected from the group consisting of —COOR₄, —C(O)NR₂R₃, —NR₂R₃, —OR₄ and —SR₄, in which:

R₂ and R₃ either represent, independently of each other, a linear, branched or cyclic, saturated or unsaturated hydrocarbon-based group containing 1 to 20, preferably 1 to 12 and better still 1 to 6 carbon atoms, optionally comprising 1 to 5 hetero atoms selected from the group consisting of N, O, S, Si and P,

or, taken together with the nitrogen atom to which they are attached, form a saturated or unsaturated hydrocarbon-based heterocycle containing 3 to 10 and preferably 4 to 6 carbon atoms and optionally 1 to 5 other hetero atoms selected from the group consisting of N, O, S, Si and P, the ring optionally being substituted with at least one linear, branched or cyclic, saturated or unsaturated hydrocarbon-based radical containing 1 to 20, preferably 1 to 15 and better still 1 to 6 carbon atoms, optionally comprising 1 to 5 hetero atoms selected from the group consisting of N, O, S, Si and P;

R₄ represents a linear, branched or cyclic, saturated or unsaturated hydrocarbon-based group containing 1 to 20, preferably 1 to 12 and better still 1 to 6 carbon atoms, which is optionally halogenated or perhalogenated especially with F, Br and/or Cl, and/or optionally comprising 1 to 5 hetero atoms selected from the group consisting of N, O, S, Si and P;

R₅ and R₆ represent, independently of each other, a group selected from the group consisting of:

(i) the saturated cyclic aminoaryl groups of formula (IIA) or (IIB):

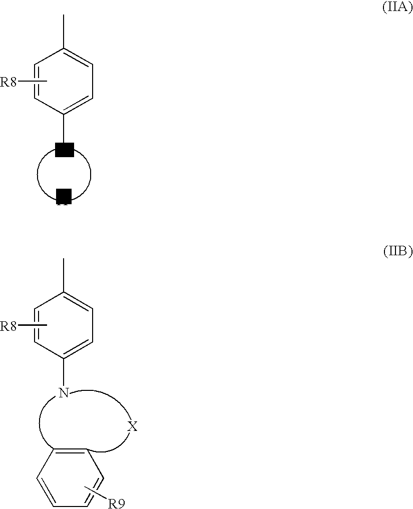

in which the ring comprising N and X is a saturated ring containing in total 3 to 30 atoms, preferably 4 to 10 and better still 5 to 8, or even 5, 6 or 7 atoms, including the nitrogen, the remainder being carbon atoms and/or hetero atoms selected from the group consisting of O, S, Si and P and/or groups selected from the group consisting of —NH and —NR with R representing a linear, branched or cyclic, saturated or unsaturated hydrocarbon-based radical containing 1 to 20, preferably 1 to 15 and better still 1 to 6 carbon atoms, optionally comprising 1 to 5 hetero atoms selected from the group consisting of N, O, S, Si and P;

(ii) the indolinoaryl groups of formula (III):

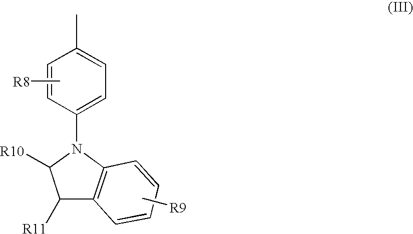

in which R₁₀ and R₁₁ represent, independently of each other, a group selected from the group consisting of (i) linear, branched or cyclic, saturated or unsaturated hydrocarbon-based groups containing 1 to 30, preferably 1 to 18 and better still 1 to 12, or even 1 to 6, carbon atoms, optionally comprising 1 to 5 hetero atoms selected from the group consisting of N, O, S, Si and P, and/or optionally halogenated or perhalogenated, especially with F, Br and/or Cl; (ii) halogen atoms, and especially F, Br and/or Cl; (iii) —CN (nitrile), —COOH (carboxylate) or —NO₂ (nitro) groups; (iv) a hydrogen atom; (v) a group selected from the group consisting of —C(O)NR₂R₃, —NR₂R₃, —OR₄ and —SR₄ with R₂, R₃ and R₄ having the meanings given above; (vi) the radicals R₁₀ and R₁₁ together possibly forming a saturated or unsaturated hydrocarbon-based ring containing in total 5 to 8 atoms (including the atoms of the indoline ring), the atoms being selected from the group consisting of C, O, S and/or NR with R representing H or a linear, branched or cyclic, saturated or unsaturated hydrocarbon-based radical containing 1 to 20 or even 1 to 12 carbon atoms, optionally comprising 1 to 5 hetero atoms selected from the group consisting of N, O, S, Si and P,

(iii) the groups of formula (IV):

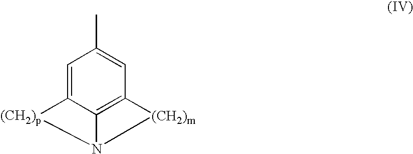

in which m and p are, independently of each other, integers ranging from 2 to 5;

(iv) the unsaturated cyclic aminoaryl groups of formula (VA), (VB) or (VC):

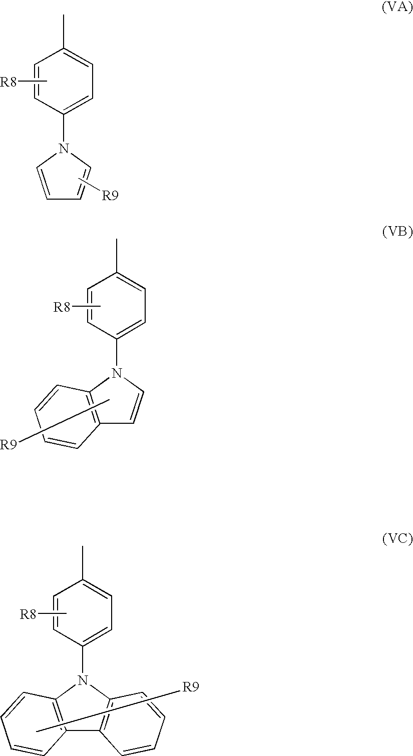

in which R₈ and R₉ represent, independently of each other, a group selected from the group consisting of (i) linear, branched or cyclic, saturated or unsaturated hydrocarbon-based groups containing 1 to 30, preferably 1 to 18 and better still 1 to 12, or even 1 to 6, carbon atoms, optionally comprising 1 to 5 hetero atoms selected from the group consisting of N, O, S, Si and P, and/or optionally halogenated or perhalogenated, especially with F, Br and/or Cl; (ii) halogen atoms, especially F, Br and/or Cl; (iii) —CN (nitrile), —COOH (carboxylate) or —NO₂ (nitro) groups; (iv) a hydrogen atom; (v) a group selected from the group consisting of —C(O)NR₂R₃, —NR₂R₃, —OR₄ and —SR₄ with R₂, R₃ and R₄ having the meanings given above;

(v) a linear, branched or cyclic, saturated or unsaturated hydrocarbon-based group containing 1 to 30, preferably 2 to 18 and better still 3 to 12 carbon atoms, optionally comprising 1 to 5 hetero atoms selected from the group consisting of N, O, S, Si and P; and especially a group selected from the group consisting of —C₆H₄—CONR₂R₃, —C₆H₄—NR₂R₃ and —C₆H₄—OR₄ with R₂, R₃ and R₄ having the meanings given above;

R₇ represents a group selected from the group consisting of:

(i) linear, branched or cyclic, saturated or unsaturated hydrocarbon-based groups containing 1 to 30, preferably 1 to 18 and better still 1 to 12 carbon atoms, optionally comprising 1 to 5 hetero atoms selected from the group consisting of N, O, S, Si and P, and/or optionally halogenated or perhalogenated, especially with F, Br and/or Cl;

(ii) halogen atoms, and especially F, Br and/or Cl;

(iii) —CN (nitrile), —COOH (carboxylate), —NO₂ (nitro), —N═N— (azo), ═NH (imino) or —CONH₂ (amide) groups;

(iv) a hydrogen atom;

(v) a group selected from the group consisting of —C(O)NR₂R₃, —NR₂R₃, —OR₄ and —SR₄ with R₂, R₃ and R₄ having the meanings given above;

(vi) the radical R₇ also possibly forming, with one of the bonds “i”, “j”, “k” or “g,h” taken with the radical R₁, or “f” taken with the radical R₁, a saturated hydrocarbon-based ring containing in total 3 to 8, preferably 4 to 7 and better still 5 or 6 carbon atoms, optionally comprising 1 to 5 hetero atoms selected from the group consisting of N, O, S, Si and P;

R′₁ represents a group selected from the group consisting of:

a hydrogen atom;

(ii) a linear, branched or cyclic, saturated or unsaturated hydrocarbon-based group containing 1 to 30, preferably 1 to 18 and better still 1 to 12 carbon atoms, optionally comprising 1 to 5 hetero atoms selected from the group consisting of N, O, S, Si and P, and/or optionally halogenated or perhalogenated, especially with F, Br and/or Cl;

(iii) a group selected from the group consisting of —C(O)NR₂R₃, —NR₂R₃, —OR₄ and —SR₄, with R₂, R₃ and R₄ having the meanings given above;

R′₂ represents a group selected from the group consisting of:

(i) linear, branched or cyclic, saturated or unsaturated hydrocarbon-based groups containing 1 to 30, preferably 1 to 18 and better still 1 to 12 carbon atoms, optionally comprising 1 to 5 hetero atoms selected from the group consisting of N, O, S, Si and P, and/or optionally halogenated or perhalogenated, especially with F, Br and/or Cl;

(ii) halogen atoms, especially F, Br and/or Cl;

(iii) —CN (nitrile), —COOH (carboxylate), —NO₂ (nitro), —N═N— (azo), ═NH (imino) or —CONH₂ (amide) groups;

(iv) a hydrogen atom;

(v) a group selected from the group consisting of —C(O)NR₂R₃, —NR₂R₃, —OR₄ and —SR₄, with R₂, R₃ and R₄ having the meanings given above.

The photochromic organic dyes according to the invention may especially correspond to one of the formulae (Ia) and (IIa) below:

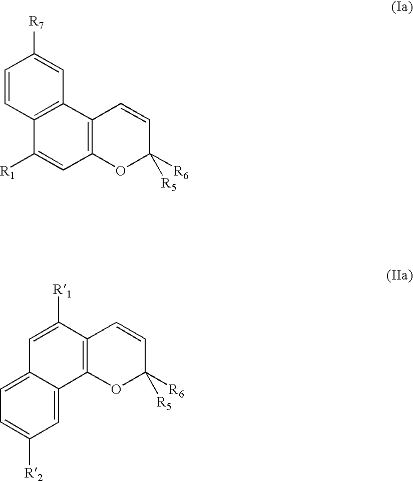

in which R₁, R₅, R₆, R₇, R′₁, and R′₂ are defined as above.

In particular, R₁ may represent a hydrogen atom; a hydrocarbon-based ring with one of the bonds “f” or “gh” and the radical R₇; or a group selected from the group consisting of —COOR₄, —NR₂R₃, —OR₄ and —SR₄, in which:

R₂ and R₃ either may represent, independently of each other, a linear, branched or cyclic, saturated or unsaturated hydrocarbon-based group containing 1 to 20, preferably 1 to 12 and better still 1 to 6 carbon atoms, optionally comprising 1 to 5 hetero atoms selected from the group consisting of N, O, S, Si and P,

or, taken together with the nitrogen atom to which they are attached, may form a saturated or unsaturated hydrocarbon-based heterocycle containing 3 to 10 and preferably 4 to 6 carbon atoms and optionally 1 to 5 other hetero atoms selected from the group consisting of N, O, S, Si and P, the ring optionally being substituted with at least one linear, branched or cyclic, saturated or unsaturated hydrocarbon-based radical containing 1 to 20, preferably 1 to 15 and better still 1 to 6 carbon atoms, optionally comprising 1 to 5 hetero atoms selected from the group consisting of N, O, S, Si and P;

R₄ may represent a linear, branched or cyclic, saturated or unsaturated hydrocarbon-based group containing 1 to 20, preferably 1 to 15 and better still 1 to 6 carbon atoms, optionally halogenated or perhalogenated, especially with F, Br and/or Cl, and/or optionally comprising 1 to 5 hetero atoms selected from the group consisting of N, O, S, Si and P.

In the present instance, R₅ and R₆ may represent, independently of each other, a group selected from the group consisting of:

the saturated cyclic aminoaryl groups of formula (IIA) or (IIB):

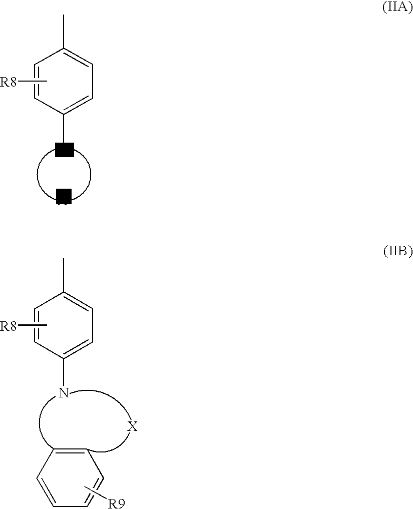

in which the ring comprising N and X is a saturated ring which contains in total 3 to 30, preferably 4 to 10 and better still 5, 6 or 7 atoms, including nitrogen, the rest being carbon atoms and/or hetero atoms selected from the group consisting of O, S, Si and P and/or groups selected from the group consisting of —NH and —NR with R representing a linear, branched or cyclic, saturated or unsaturated hydrocarbon-based radical containing 1 to 20, preferably 1 to 15 and better still 1 to 6 carbon atoms, optionally comprising 1 to 5 hetero atoms selected from the group consisting of N, O, S, Si and P;

a linear, branched or cyclic, saturated or unsaturated hydrocarbon-based group containing 1 to 30, preferably 2 to 18 and better still 3 to 12 carbon atoms, optionally comprising 1 to 5 hetero atoms selected from the group consisting of N, O, S, Si and P; and especially a group selected from the group consisting of —C₆H₄—CONR₂R₃, —C₆H₄—NR₂R₃ and —C₆H₄—OR₄ with R₂, R₃ and R₄ having the meanings given above.

In particular, R₇ may represent a group selected from the group consisting of:

(i) linear, branched or cyclic, saturated or unsaturated hydrocarbon-based groups containing 1 to 30, preferably 1 to 18 and better still 1 to 12 carbon atoms, optionally comprising 1 to 5 hetero atoms selected from the group consisting of N, O, S, Si and P, and/or optionally halogenated or perhalogenated, especially with F, Br and/or Cl;

(ii) halogen atoms, especially F, Br and/or Cl;

(iii) —CN (nitrile), —COOH (carboxylate), —NO₂ (nitro), —N═N— (azo), ═NH (imino) or —CONH₂ (amide) groups;

(iv) a hydrogen atom;

(v) a group selected from the group consisting of —NR₂R₃, —OR₄ and —SR₄, with R₂, R₃ and R₄ having the meanings given above;

(vi) the radical R₇ also possibly forming, with one of the bonds “i”, “j”, “k” or “g,h” taken with the radical R₁, or “f” taken with the radical R₁, a saturated hydrocarbon-based ring containing in total 3 to 8, preferably 4 to 7 and better still 5 or 6 carbon atoms, optionally comprising 1 to 5 hetero atoms selected from the group consisting of N, O, S, Si and P.

In particular, R′₁ may represent hydrogen or a linear, branched or cyclic, saturated or unsaturated hydrocarbon-based group containing 1 to 30, preferably 1 to 18 and better still 1 to 12 carbon atoms, optionally comprising 1 to 5 hetero atoms selected from the group consisting of N, O, S, Si and P, and/or optionally halogenated or perhalogenated, especially with F, Br and/or Cl.

In particular, R′₂ represents hydrogen or a group selected from the group consisting of —NO₂, —NR₂R₃ and —C(O)NR₂R₃, in which R₂ and R₃ either represent, independently of each other, a linear, branched or cyclic, saturated or unsaturated hydrocarbon-based group containing 1 to 20, preferably 1 to 12 and better still 1 to 6 carbon atoms, optionally comprising 1 to 5 hetero atoms selected from the group consisting of N, O, S, Si and P; or, taken together with the nitrogen atom to which they are attached, form a saturated or unsaturated hydrocarbon-based heterocycle containing 3 to 10 and preferably 4 to 6 carbon atoms and optionally 1 to 5 other hetero atoms selected from the group consisting of N, O, S, Si and P, the ring optionally being substituted with at least one linear, branched or cyclic, saturated or unsaturated hydrocarbon-based radical containing 1 to 20, preferably 1 to 15 and better still 1 to 6 carbon atoms, optionally comprising 1 to 5 hetero atoms selected from the group consisting of N, O, S, Si and P.

Mention may be made more particularly of the photochromic dyes of formula (I) or (Ia) for which:

R₁ represents hydrogen; or a group —COOR with R being a saturated hydrocarbon-based radical containing 1 to 12 and preferably 1 to 6 carbon atoms, and especially a methyl or ethyl radical; or a group

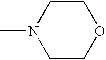

and/or

R₅ and R₆ represent, independently of each other, either (i) a group of formula (IIA):

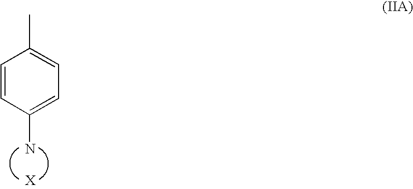

in which the ring comprising N and X is a saturated ring containing in total 4 to 7 atoms and especially 5 or 6 atoms, including nitrogen, and especially 3 to 5 carbon atoms and 0 or 1 oxygen atom; and in particular a group of formula:

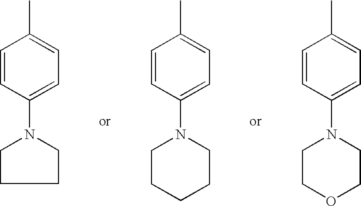

or (ii) a linear, branched or cyclic, saturated or unsaturated hydrocarbon-based group containing 5 to 14 and preferably 6 to 10 carbon atoms, optionally comprising 1 or 2 hetero atoms selected from the group consisting of N, O and S;

in particular a group

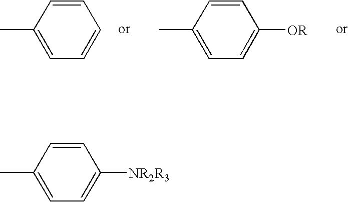

in which R is a saturated hydrocarbon-based radical containing 1 to 12 and preferably 1 to 6 carbon atoms, and especially a methyl or ethyl radical; and R₂ and R₃ are, independently of each other, a linear, branched or cyclic, saturated or unsaturated hydrocarbon-based group containing 1 to 20, preferably 1 to 15 and better still 1 to 6 carbon atoms, optionally comprising 1 to 5 hetero atoms selected from the group consisting of N, O, S, Si and P;

and/or

R₇ represents a hydrogen atom or a group —NR₂R₃, with R₂ and R₃ representing, independently of each other, a linear or branched, saturated hydrocarbon-based group containing 1 to 12 and preferably 1 to 6 carbon atoms, and especially a methyl and/or ethyl group.

Mention may also be made of the organic dyes of formula (II) or (IIa) for which:

R′₁ represents hydrogen or a group —COOR with R being a saturated hydrocarbon-based radical containing 1 to 12 and preferably 1 to 6 carbon atoms, and especially a methyl or ethyl radical;

and/or

R₅ and R₆ represent, independently of each other, either (i) a group of formula (IIA):

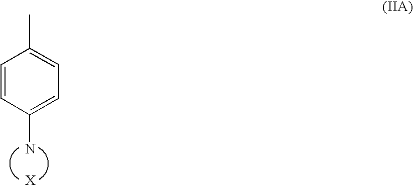

in which the ring comprising N and X is a saturated ring containing in total 4 to 7 atoms and especially 5 to 6 atoms, including nitrogen, and especially 4 to 5 carbon atoms and 0 or 1 oxygen atom; and in particular a group of formula:

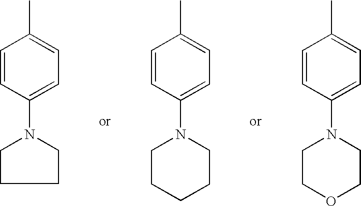

or (ii) a linear, branched or cyclic, saturated or unsaturated hydrocarbon-based group containing 5 to 14 and preferably 6 to 10 carbon atoms, optionally comprising 1 or 2 hetero atoms selected from the group consisting of N, O and S;

in particular a group:

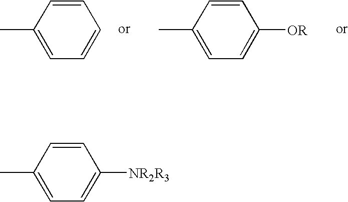

in which R is a saturated hydrocarbon-based radical containing 1 to 12 and preferably 1 to 6 carbon atoms, and especially a methyl or ethyl radical; and R₂ and R₃ are, independently of each other, a linear, branched or cyclic, saturated or unsaturated hydrocarbon-based group containing 1 to 20, preferably 1 to 15 and better still 1 to 6 carbon atoms, optionally comprising 1 to 5 hetero atoms selected from the group consisting of N, O, S, Si and P;

and/or

R′₂ represents hydrogen or a group —NR′R″, with R′ and R″, which may be identical or different, representing a linear or branched, saturated hydrocarbon-based group containing 1 to 12 and preferably 1 to 6 carbon atoms, and especially a methyl and/or ethyl group; or a group

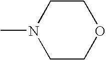

The derivatives as defined above that have photochromic activity are most particularly suitable for the invention.

Non-limiting illustrations of dyes of naphthopyran type that may be mentioned include those described in patent applications WO 94/22850, WO 98/45281 and WO 00/18755.

More specifically, the following compounds are most particularly suitable for the invention:

3,3-di(4-methoxyphenyl)-6-morpholino-3H-naphtho[2,1-b]pyran of formula:

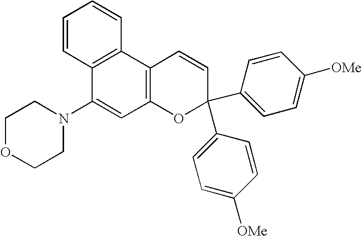

3-phenyl-3-(4-morpholinophenyl)-6-morpholino-3H-naphtho[2,1-b]pyran of formula:

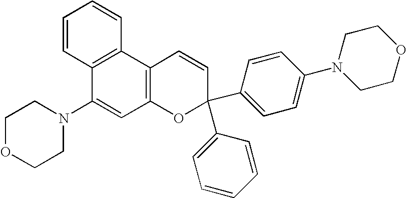

3-phenyl-3-(4-piperidinophenyl)-6-morpholino-3H-naphtho[2,1-b]pyran of formula:

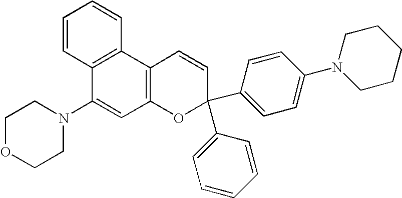

3-phenyl-3-(4-piperidinophenyl)-6-carboxymethyl-9-N-dimethyl-3H-naphtho[2,1-b]pyran of formula:

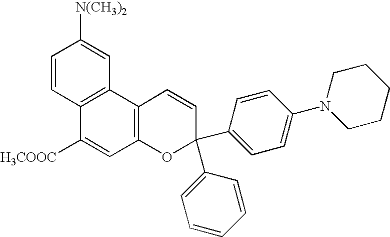

2-phenyl-2-(4-piperidinophenyl)-5-carboxymethyl-9-N-dimethyl-2H-naphtho[1,2-b]pyran of formula:

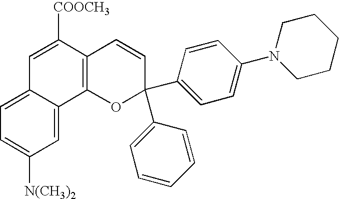

and mixtures thereof.

Goniochromatic Coloring Agents

For the purposes of the present invention, the term “goniochromatic coloring agent” denotes a coloring agent for obtaining, when the composition containing it is spread onto a support, a color path in the a*b* plane of the 1976 CIE (International Commission on Illumination) colorimetric space that corresponds to a variation Dh of the hue angle h of at least 20° when the angle of observation relative to the normal is varied between 0° and 80°, for an angle of light incidence of 45°.

The color path may be measured, for example, using an Instrument Systems Spectrogonioreflectometer of reference GON 360 Goniometer, after the cosmetic composition has been spread in fluid form to a thickness of 300 μm using an automatic spreader onto an Erichsen contrast card of reference Typ 24/5, the measurement being performed on the black background of the card.

The goniochromatic coloring agent may be present, for example, in any amount including in an amount that can range, on a weight basis relative to the total weight of the composition containing it, from 0.1% to 60%, or even from 1% to 20% or from 2% to 15%, and better still from 2% to 10%, especially for a composition intended to be applied to the lips. A nail varnish composition may contain, for example, between 0.1% and 5% of goniochromatic coloring agent; a foundation may contain from 10% to 15% and a lipstick may contain from 2% to 8% by weight.

The goniochromatic coloring agent may be chosen so as to produce a relatively large color change with the angle of observation.

The goniochromatic coloring agent may be chosen, for example, from multilayer interference structures and liquid-crystal coloring agents.

In the case of a multilayer structure, it may comprise, for example, at least two layers, each layer, optionally independently of the other layer(s), being made, for example, from at least one material selected from the group consisting of the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys and polymers, and combinations thereof.

As regards the liquid-crystal goniochromatic particles that may be used in the composition according to the invention, they may especially be based on a polymer that may be obtained by polymerization of a monomer mixture comprising:

a) at least a first monomer A of formula (I) Y1-A1-M1-A2-Y2 in which

i) Y1 and Y2, which may be identical or different, represent a polymerizable group selected from the group consisting of acrylate or methacrylate groups, an epoxy group and an isocyanate, hydroxyl, vinyl ether (—O—CH═CH₂) or vinyl ester (—CO—O—CH═CH₂) group, and

ii) A1 and A2, which may be identical or different, represent a group of formula —CnH₂n—, in which n is an integer ranging from 0 to 20 and one or more methylene groups of the group —CnH₂n— possibly being replaced with one or more oxygen atoms, and

iii) M1 denotes a group of general formula (I′) —R₁—X₁—R₂—X₂—R₃—X₃—R₄—, in which R₁, R₂, R₃ and R₄, which may be identical or different, denote a divalent group selected from the group consisting of —O—, —COO—, —CONH—, —CO—, —S—, —C—C—, —CH≡CH—, —N═N—, —N═N(O)— and —R₂—X₂—R₃— or —R₂—X₂— or —R₂—X₂—R₃—X₃— also possibly being a single covalent bond, and X₁, X₂ and X₃ are identical or different groups selected from the group consisting of 1,4-phenylene and 1,4-cyclohexylene groups, the arylene or heteroarylene groups having an aryl nucleus containing from 6 to 10 atoms optionally substituted with B1 and/or B2 and/or B3, the heteroarylene containing from 1 to 3 hetero atoms selected from the group consisting of O, N and S atoms, cycloalkylene groups containing from 3 to 10 carbon atoms optionally substituted with -B1 and/or -B2 and/or -B3, -B1, -B2, and -B3, which may be identical or different, being selected from the group consisting of C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups, and alkyl, alkoxy or alkylthio groups containing from 1 to 20 carbon atoms, interrupted with one or more oxygen atoms(s) or one or more sulphur atom(s) or one or more ester group(s),

and

b) at least a second monomer B, which is chiral, of formula (II) V1-A′1-W1-Z-W2-A′2-V2, in which

i) V1 and V2, which may be identical or different, denote a group selected from the group consisting of an acrylate or methacrylate group, an epoxy group, a vinyl ether or vinyl ester group, an isocyanate group, a C₁-C₂₀ alkyl, a C₁-C₂₀ alkoxy, a C₁-C₂₀ alkylthio, a (C₁-C₂₀)alkoxycarbonyl, a (C₁-C₂₀)alkylthiocarbonyl, —OH, —F, —Cl, —Br, —I, —CN, —NO₂, formyl and acetyl, and alkyl, alkoxy or alkylthio groups containing from 1 to 20 carbon atoms and interrupted with one or more oxygen atom(s) or one or more sulphur atom(s) or one or more ester (—CO—O—) group(s),

and at least V1 or V2 denotes a polymerizable group selected from the group consisting of acrylate or methacrylate groups, an epoxy group and an isocyanate, hydroxyl, vinyl ether (—O—CH═CH₂) or vinyl ester (—CO—O—CH═CH₂) group,

ii) A′1 and A′2, which may be identical or different, represent a group of formula —CₙH₂ₙ—, in which n is an integer ranging from 0 to 20 and one or more methylene groups of the group CₙH₂ₙ possibly being replaced with one or more oxygen atoms, and

iii) W1 and W2 denote a divalent group of general formula R′₁—X′₁—R′₂—X′₂—R′₃— in which R′₁, R′₂ and R′₃, which may be identical or different, denote a divalent group selected from the group consisting of —O—, —COO—, —CONH—, —CO—, —S—, —C≡C—, —CH═CH—, —N═N—, and —N═N(O)—, and R′₁, R′₂, R′₃ or R′₂—X′₂ may also be a single covalent bond, and X′₁ and X′₂ are identical or different groups selected from the group consisting of 1,4-phenylene and 1,4-cyclohexylene groups, the arylene or heteroarylene groups having an aryl nucleus containing from 6 to 10 atoms optionally substituted with B′1 and/or B′2 and/or B′3, the heteroarylene containing from 1 to 3 hetero atoms selected from the group consisting of O, N and S atoms, the cycloalkylene groups containing from 3 to 10 carbon atoms optionally substituted with -B′1 and/or -B′2 and/or -B′3, -B′1, -B′2, and -B′3, which may be identical or different, being selected from the group consisting of C₁-C₂₀ alkyl, C₁-C₂₀ alkoxy, C₁-C₂₀ alkylthio, (C₁-C₂₀)alkylcarbonyl, (C₁-C₂₀)alkoxycarbonyl, (C₁-C₂₀)alkylthiocarbonyl, —OH, —F, —Cl, —Br, —I, —CN, —NO₂, formyl and acetyl groups, and alkyl, alkoxy or alkylthio groups containing from 1 to 20 carbon atoms, interrupted with one or more oxygen atoms(s) or one or more sulphur atom(s) or one or more ester group(s),

and Z denotes a divalent chiral group comprising at least 4 carbon atoms, especially from 4 to 20 carbon atoms and better still from 4 to 10 carbon atoms (the divalent chiral group comprising at least one asymmetric carbon, especially one or two asymmetric carbons and in particular two asymmetric carbons) and in particular a divalent chiral group derived from the family of dianhydrohexites, hexoses, pentoses, binaphthyl derivatives (binaphthyl groups), biphenyl derivatives (biphenyl groups), tartaric acid derivatives and optically active glycols.

The liquid-crystal polymer is preferably obtained by polymerization of a mixture of monomers comprising:

a) at least a first monomer A of formula (I) Y1-A1-M1-A2-Y2

in which

i) Y1 and Y2, which may be identical or different, represent an acrylate or methacrylate group, and preferably an acrylate group;

ii) A1 and A2, which may identical or different, represent a group of formula —CnH₂n—, in which n is an integer ranging from 1 to 20, preferably from 2 to 6 and better still equal to 4;

iii) M1 denotes a group of general formula (I′) —R₁—X₁—R₂—X₂—R₃—X₃—R₄—, in which R₁ and R₄ denote —O—, and R₂ and R₃ denote —COO—,

and X₁, X₂ and X₃ are a 1,4-phenylene group, the carbonyl group —CO— of R₂ and of R₃, respectively, being linked to the group X₁ or X₃, respectively,

and

b) at least a second monomer B, which is chiral, of formula (II) V1-W1-Z-W2-V2, in which

i) V1 denotes an acrylate or methacrylate group, and preferably an acrylate group, and V2 denotes a C₁-C₂₀ alkyl, a C₁-C₂₀ alkoxy, a (C₁-C₂₀)alkoxycarbonyl or —OH group, and preferably denotes a C₁-C₂₀ and especially C₁-C₄ alkoxy group, and in particular a methoxy group;

ii) W1 represents a divalent group of formula —X′1—CO—O—,

W2 represents a divalent group of formula —O—CO—X′1—,

in which X′1 denotes a 1,4-phenylene group,

and Z denotes a chiral group containing two bonds, derived from the dianhydrohexite group, in particular a divalent radical of formula:

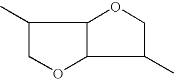

Preferably, the monomer mixture comprises from 70% to 99% by weight of monomer A and from 1% to 30% by weight of monomer B, and better still comprises from 90% to 95% by weight of monomer A and from 5% to 10% by weight of monomer B, relative to the total weight of monomer A and monomer B.

Preferably, the concentration of the polymerizable groups present in the mixture of monomer A and of monomer B (polymerizable groups Y1 and Y2 of monomer A and polymerizable groups V1 and V2 of monomer B) ranges from 3.2 to 15 mmol/g.

According to one particular embodiment of the invention, the liquid-crystal polymer is such that the mixture of monomer A and of monomer B comprises polymerizable groups, at least 90% of which are present in monomers containing at least two polymerizable groups, in a concentration ranging from 3.2 to 15 mmol/g.

In particular, the liquid-crystal polymer essentially comprises or consists of a mixture of the monomers A and B defined above.

The liquid-crystal polymer especially has a helix pitch of greater than 450 nm, especially ranging from 455 mn to 5 000 nm and in particular ranging from 455 nm to 1 000 nm, or even ranging from 455 nm to 650 nm.

The monomer A may have a weight-average molecular weight ranging from 150 to 800 and especially ranging from 460 to 625. The monomer A may be in particular an unsubstituted hydroquinone dibenzoate derivative.

The monomer B may have a weight-average molecular weight ranging from 500 to 1 000 and especially ranging from 500 to 700.

The liquid-crystal polymer may have a weight-average molecular weight of less than 625.

The liquid-crystal polymer defined above may be prepared according to the processes known in the prior art, such as those described in documents U.S. Pat. No. 5,362,315 and U.S. Pat. No. 5,807,497, starting with the monomer mixture described above.

The polymerization of the directed monomer mixture may be performed, in an already known manner, for example via a free-radical route with use of commercial thermal initiators, using electron beams or UV light in combination with commercial photoinitiators, or alternatively via addition reactions or condensation reactions.

The crosslinking of the monomer mixtures, in the chiral structural form, preferably takes place via a polyreaction which, depending on the type of polymerizable, polycondensable or polyadditionable groups, proceeds in the form of a free-radical, ionic or metal-catalysed polymerization, or a polycondensation reaction or a polyaddition reaction.

The initiation of the free-radical polymerization may be performed using corresponding initiators or via UV irradiation, using commercial photoinitiators or via high-energy irradiation, such as an electron beam. An advantage of thermal polymerization of the radicals or of polymerization via curing with electron beams lies in the fact that a light stabilizer, such as a UV (UVA) absorber or free-radical scavengers (HALS), may also be added to the polymerizable mixture to stabilize the pigments or the resulting films against UV light, for example for external applications, without resulting in losses in the polymerization conversion, as is the case during UV curing, due to the UVA-screening effect of the photoinitiator. There is thus no reduction in the crosslinking density.

If the curing of the films is performed with peroxide or with an electron beam, the monomer mixture preferably contains commercial light stabilizers, such as UV absorbers or free-radical scavengers, in an overall concentration of from 0.5% to 5% by weight.

Besides the photostabilizers, the monomer mixtures may also contain other common additives for inhibiting oxidation or inhibiting polymerization, or additives for improving the Theological properties. Furthermore, absorbent fillers, such as pigments or soot, and also fluorescent colorants or pigments, may be contained.

The film obtained after polymerization is then ground into particles, especially in the form of platelets.

Preferably, the particles of liquid-crystal polymer have a larger size ranging from 1 μm to 3 mm and preferably ranging from 30 μm to 500 μm. These particles are advantageously in the form of platelets.

The particles may be separated (sorted) via a process with grain size selectivity.

Such polymers and their particles are described in patent application EP-A-1 046 692.

Liquid-crystal polymer particles that may especially be used include those known under the CTFA name Polyacrylate-4 and sold under the names “Helicone® HC Sapphire”, “Helicone® HC Scarabeus”, “Helicone® HC Jade”, “Helicone® HC Maple”, “Helicone® HC XL Sapphire”, “Helicone® HC XL Scarabeus”, “Helicone® HC XL Jade” and “Helicone® HC XL Maple” by the company Wacker.

The particles of the liquid-crystal polymer may be present in the product according to the invention in a content ranging from 0.01% to 99% by weight, especially ranging from 0.1% to 60% by weight, in particular ranging from 1% to 30% by weight and even ranging from 5% to 15% by weight, relative to the total weight of the composition containing them.

Other Dyes

For the purposes of the present invention, this term is intended to cover non-goniochromatic dyes and/or dyes that are different from the dyes referred to as “first dyes” according to the invention. They are more particularly any compound selected from the group consisting of liposoluble and water-soluble monochromatic dyes, nacres and reflective particles.

Liposoluble and Water-Soluble Monochromatic Dyes

The first and/or the second composition(s) may also comprise at least one monochromatic dye, especially a natural organic dye such as cochineal carmine, and/or a synthetic dye such as halo acid dyes, azo dyes or anthraquinone dyes. Mention may also be made of mineral dyes such as copper sulphate or iron sulphate. Mention may also be made of Sudan brown, Sudan red and annatto, and also beetroot juice, carotene and methylene blue.

The dye may be present in the composition under consideration alone or as a mixture, in a proportion of from 0.001% to 15% by weight, especially from 0.01% to 5% by weight and in particular from 0.1% to 2% by weight, relative to the total weight of the composition.

Pigments

The term “pigments” should be understood as meaning white or colored, mineral or organic particles intended to color and/or opacify the composition.

Pigments may be present in the first and/or second composition(s) especially in a proportion of from 0.01% to 25% by weight and preferably in a proportion from 3% to 10% by weight of the composition under consideration.

They may be white or colored, mineral or organic, and of standard or nanometric size. They may be in the form of powder or of pigmentary paste. Mention may be made of titanium oxide, zirconium oxide or cerium oxide, and also zinc oxide, iron oxide, or chromium oxide, ferric blue, chromium hydrate, carbon black, ultramarines (aluminosilicate polysulphides), manganese pyrophosphate and certain metal powders such as silver or aluminium powder. Mention may also be made of the D&C pigments and lakes commonly used to give the lips and the skin a makeup effect, which are calcium, barium, aluminium, strontium or zirconium salts.

Nacres

The term “nacres” should be understood as meaning iridescent particles that reflect light, which are produced especially by certain molluscs in their shell, or alternatively which are synthesized.

Nacres may be present in the first and/or second composition(s) especially in a proportion of from 0.01% to 20% by weight and preferably in a content of about from 3% to 10% by weight relative to the total weight of the composition under consideration. Among the nacres that may be envisaged, mention may be made of natural nacre, mica coated with titanium oxide, with iron oxide, with natural pigment or with bismuth oxychloride, and also colored titanium mica.

Reflective Particles

For the purposes of the present invention, the term “reflective particles” denotes particles for which the size, the structure, in particular the thickness of the layer(s) of which they are made and the physical and chemical natures thereof, and the surface state, allow them to reflect incident light with a sufficient intensity to be able to create at the surface of the claimed composition, when this composition is applied to the support to be made up, highlight points that are visible to the naked eye, i.e. brighter points that contrast with their environment and appear to shine.

Reflective particles may be present in the first and/or second composition(s) by being uniformly dispersed, for example in a content ranging from 0.1% to 20%, especially from 1% to 15% by weight and in particular from 1% to 10% by weight, for example about 2%, relative to the total weight of the composition, especially for a composition intended to be applied to the lips.

Reflective particles that allow a metallic reflection of the incident light are most particularly suitable. This is the case especially when the reflective particles, irrespective of their shape, allow reflection on a layer of a metal, for example of silver. Such particles are found to be relatively neutral with respect to the color of the composition.

Reflective particles that may be used in the invention, with a metallic or white glint, can, for example, reflect light in all the components of the visible region without significantly absorbing one or more wavelengths. The spectral reflectance of these reflective particles may be, for example, greater than 70% in the 400-700 nm range, and in particular at least 80%, or even 90% or 95%.

The light reflected by the reflective particles may be non-iridescent, especially in the case of a metallic glint.

The reflective particles, irrespective of their shape, may or may not have a multilayer structure, and, in the case of a multilayer structure, for example at least one layer of uniform thickness, especially of a reflective material.

The reflective material may include a layer of metal or of a metallic compound.

Glass particles coated with a metallic layer are described especially in documents JP-A-09 188 830, JP-A-10 158 450, JP-A-10 158 541, JP-A-07 258 460 and JP-A-05 017 710.

The first and/or second composition(s) may also comprise at least one compound that is photochromic but different from those under consideration according to the invention, i.e. characterized generally by a ΔE value <5 and, where appropriate, insolubility in the composition under consideration. Mention may be made especially of mineral photochromic compounds and more particularly doped aluminosilicates such as halogen-doped sodalite, or metal oxides or hydrates such as titanium oxides rendered photochromic with the aid of a metal selected from the group consisting of iron, chromium, copper, nickel, manganese, cobalt or molybdenum, in elemental form or in the form of a salt such as a sulphate, a chlorate, a nitrate or an acetate. Such a photochromic compound may be incorporated into the first and/or second composition(s) in an amount of from 0.001% to 20% by weight and especially in an amount of from 0.1% to 10% by weight relative to the total weight of the composition under consideration.

Physiologically Acceptable Medium

The physiologically acceptable media for each of the compositions of which the product according to the invention is composed may be adapted to the nature of the makeup to be applied, the nature of the surface to be made up or treated onto which the product is to be applied, and also to the form in which the product is intended to be packaged, especially solid or fluid at room temperature and atmospheric pressure.

The first and/or second composition(s) of the product according to the invention may comprise at least one aqueous phase and/or at least one fatty phase.

The first and/or second composition(s) of the product according to the invention may be anhydrous, or may also comprise an aqueous phase, which may comprise water, a floral water such as cornflower water and/or a mineral water such as eau de Vittel, eau de Lucas or eau de La Roche Posay.

The aqueous phase may comprise from 0.1% to 14% by weight, relative to the total weight of the aqueous phase, of a C₂-C₆ monoalcohol, for instance ethanol, propanol, butanol, isopropanol or isobutanol.

Oils

Generally, the first and/or second composition(s) of the product according to the invention comprise(s) at least one oily phase, which may comprise one or more cosmetically acceptable oils.

According to the invention, the term “cosmetically acceptable oil” means any fatty substance that is liquid at 25° C. and 1 atm, with a molecular weight of greater than or equal to 160, especially between 170 and 106, or even between 200 and 5 105, which is compatible with application to the skin, mucous membranes (the lips) and/or the integuments (nails, eyelashes, eyebrows or hair).

Preferably, the oily phase is macroscopically homogeneous, i.e. homogeneous to the naked eye.

The oily phase may comprise one or more oils, which may be polar or apolar, volatile or non-volatile and, preferably, hydrocarbon-based.

The term “polar oil” means an oil composed of chemical compounds comprising at least one polar group. Polar groups are well known to those skilled in the art: they may especially be groups of alcohol, ester or carboxylic acid type.

In particular, the polar oils according to the invention may be defined as having a mean solubility parameter δₐ according to the Hansen solubility space, at 25° C., of: δₐ≧5.0 (J/cm³)½.

The polar oils comprise the relatively polar oils that have a mean solubility parameter at 25° C. of: 5.0≦δₐ≦7.0 (J/cm³)½, and the definitely polar oils, which have a mean solubility parameter at 25° C. of: δₐ>7.0 (J/cm³)½.

Similarly, for the purposes of the invention, the apolar oils have a mean solubility parameter δₐ according to the Hansen solubility space, at 25° C., of: 0≦δₐ<5.0 (J/cm³)½.

For the purposes of the invention, the apolar oils comprise the definitely apolar oils (δₐ=0) and the sparingly polar oils, which have a mean solubility parameter at 25° C. of: 0<δₐ<5.0 (J/cm³)½.

Thus, the higher the value of δₐ, the higher the polarity of the oil.

The definition and calculation of the solubility parameters in the three-dimensional Hansen solubility space are described in the article by C. M. Hansen: “The three dimensional solubility parameters”, J. Paint Technol. 39, 105 (1967).

According to this Hansen space:

δ_{D} characterizes the London dispersion forces derived from the formation of dipoles induced during molecular impacts;

δₚ characterizes the Debye interaction forces between permanent dipoles and also the Keesom interaction forces between induced dipoles and permanent dipoles;

δₕ characterizes the specific interaction forces (such as hydrogen bonding, acid/base, donor/acceptor, etc.);

δₐ is determined by the equation: δₐ=(δₚ²+δₕ²)1/2

The parameters δₚ, δₕ, δ_{D} and δₐ are expressed in (J/cm³)½.

When the oily phase is a mixture of different oils, the solubility parameters of the mixture are determined from those of the compounds taken separately, according to the following relationships:

$${δ}_{D mel} = \frac{Σ}{i} x i δ D i ; δ = \frac{Σ}{i} x i δ p i and δ = \frac{Σ}{i} x i δ h i$$

$${δ}_{amel} = ( {δ}_{pmel}^{2} + {δ}_{hmel}^{2} ) 1 / 2$$

in which xi represents the volume fraction of the compound i in the mixture.

It is within the capability of a person skilled in the art to determine the amounts of each oil in order to obtain an oily phase that satisfies the desired criteria.

According to a first embodiment of the invention, the oily phase is polar and may mainly, or even exclusively, comprise one or more polar oils (relatively or definitely polar) as a mixture, which may thus represent 5% to 100% by weight, especially from 10% to 90% or even from 15% to 60%, and in particular from 20% to 50% by weight, relative to the total weight of the oily phase.

According to this embodiment, the polar oily phase has a mean solubility parameter δₐ according to the Hansen solubility space, at 25° C., of greater than or equal to 5.0 (J/cm³)½, especially greater than or equal to 5.3 or even greater than or equal to 5.5, and better still greater than or equal to 6.0 (J/cm³)½ or even greater than or equal to 7.0 (J/cm³)½.

According to a second particular embodiment of the invention, the oily phase is apolar and may comprise 5% to 100% by weight, especially 10 to 90%, or even 15 to 60% and better still 20 to 50% by weight of one or more apolar (apolar or sparingly polar) oils; it has a mean solubility parameter δₐ according to the Hansen solubility space, at 25° C., of less than 5.0, especially less than or equal to 4.9, better still less than or equal to 4.5 and even better still less than or equal to 4.0 (J/cm³)½.

The oils that may be used in the oily phase may be chosen, alone or as a mixture, from volatile or non-volatile oils of animal, plant, mineral or synthetic origin.

The term “volatile oils” means oils that have, at 25° C., a vapour pressure of between 0.02 and 300 mmHg (i.e. 2.66 to 40 000 Pa). Preferably, volatile oils with a flash point of about 30-100° C. are used.

Mention may be made especially of:

animal or plant oils, especially formed from fatty acid esters of polyols, in particular liquid triglycerides, for example sunflower oil, maize oil, soybean oil, marrow oil, grapeseed oil, sesame seed oil, hazelnut oil, apricot oil, almond oil or avocado oil; fish oils or glyceryl tricaprocaprylate, or oils of plant or animal origin of formula R₁COOR₂ in which R₁ represents a higher fatty acid residue containing from 7 to 19 carbon atoms and R₂ represents a branched hydrocarbon-based chain containing from 3 to 20 carbon atoms, for example Purcellin oil; liquid paraffin, liquid petroleum jelly, beauty-leaf oil, macadamia oil, rapeseed oil, coconut oil, groundnut oil, palm oil, castor oil, jojoba oil, olive oil or cereal germ oil; shea butter oil; perhydrosqualene;

synthetic esters and ethers, especially of fatty acids, for instance the oils of formula R₁COOR₂ in which R₁ represents a higher fatty acid residue containing from 7 to 29 carbon atoms and R₂ represents a hydrocarbon-based chain containing from 3 to 30 carbon atoms, for instance isopropyl myristate, 2-ethylhexyl palmitate, 2-octyldodecyl stearate, 2-octyldodecyl erucate, isononyl isononanoate or isostearyl isostearate; hydroxylated esters, for instance isostearyl lactate, octyl hydroxystearate, octyldodecyl hydroxystearate, diisostearyl malate, triisocetyl citrate, and fatty alkyl heptanoates, octanoates and decanoates; polyol esters, for instance propylene glycol dioctanoate, neopentyl glycol diheptanoate or diethylene glycol diisononanoate; pentaerythritol esters, for instance pentaerythrityl tetraisostearate; esters of the type such as tridecyl trimellitate;

fatty alcohols containing from 12 to 26 carbon atoms, for instance octyldodecanol, 2-butyloctanol, 2-hexyldecanol, 2-undecylpentadecanol or oleyl alcohol;

linear or branched hydrocarbons of mineral or synthetic origin, such as liquid paraffins and derivatives thereof, petroleum jelly, polydecenes, and hydrogenated polyisobutene such as parleam; isoparaffins, for instance isohexadecane and isodecane;

glycerides and especially acetylglycerides or triglycerides of fatty acids containing 4 to 10 carbon atoms, for instance heptanoic, octanoic and capric/caprylic acid triglycerides,

and mixtures thereof.

Among the polar oils that are particularly preferred, mention may be made of octyldodecanol, hexyldecanol, octyldecanol, oleyl alcohol, castor oil, diisostearyl malate, glyceryl triheptanoate, glyceryl trioctanoate, capric/caprylic acid triglyceride, triisononanoin and tridecyl trimellitate, and mixtures thereof.

Among the apolar oils that are particularly preferred, mention may be made of aliphatic hydrocarbons, especially of C₆-C₄₀, for instance volatile liquid paraffins, such as isohexadecane or isododecane, or non-volatile liquid paraffins, and derivatives thereof; petroleum jelly, hydrogenated or non-hydrogenated polydecenes, hydrogenated polyisobutene such as parleam oil, squalane, polybutylenes and isononyl isononanoate; fluoro oils and especially perfluoro oils, and mixtures thereof.

Mention may be made especially of the following oils:

| | δₐ (J/cm³)^{1/2} |
|---|---|
| Castor oil | 9.09 |
| 2-Hexyldecanol | 8.55 |
| Oleyl alcohol | 8.17 |
| Octyldodecanol | 7.69 |
| Heptanoic acid triglyceride | 7.29 |
| Diisostearyl malate | 7.19 |
| Octanoic acid triglyceride | 6.87 |
| Capric/caprylic acid triglyceride | 6.69 |
| Triisononanoin | 6.54 |
| Tridecyl trimellitate | 5.35 |
| Isononyl isononanoate | 4.87 |
| Hydrogenated polyisobutene | 0 |
| Isododecane | 0 |

The oily phase is preferably chosen such that the photochromic dye(s) are, if not soluble, then at least dispersible as they are, i.e. without an associated surface treatment.

The oily phase is preferably included in an amount of from 1% to 99% by weight, especially 10% to 90% by weight and preferably 15% to 80% by weight, relative to the total weight of the composition under consideration.

The first and/or second composition(s) of the product according to the invention may also comprise fatty substances other than the above oils, which may be chosen by a person skilled in the art on the basis of his general knowledge, so as to give the final product the desired properties, for example in terms of consistency and/or texture.

These additional fatty substances may be waxes, gums and/or pasty fatty substances of animal, plant, mineral or synthetic origin, and also mixtures thereof.

For the purposes of the present invention, a wax is a lipophilic fatty compound, which is solid at room temperature (25° C.), with a reversible solid/liquid change of state, which has a melting point of greater than 40° C. and which may be up to 200° C., which generally has a hardness of greater than 0.5 MPa and which has an anisotropic crystal organization in the solid state.

Mention may be made especially of waxes of animal, plant, mineral or synthetic origin, such as microcrystalline waxes, paraffin wax, petrolatum, petroleum jelly, ozokerite or montan wax; beeswax, lanolin wax and derivatives thereof; candelilla wax, ouricury wax, carnauba wax, Japan wax, cocoa butter, cork fibre wax or sugar cane wax, lignite wax, rice bran wax, fir tree wax and cotton wax; hydrogenated oils with a melting point of greater than (about) 40° C., for instance hydrogenated jojoba oil; fatty esters and glycerides that are solid at 25° C.; polyethylene waxes and the waxes obtained by Fischer-Tropsch synthesis; hydrogenated oils that are solid at 25° C.; lanolins.

The pasty fatty substances generally have a melting point of between 25 and 60° C. and preferably between 30 and 45° C., and/or a hardness ranging from 0.001 to 0.5 MPa and preferably between 0.005 and 0.4 MPa. Mention may be made especially of lanolins and derivatives thereof, or cholesterol esters.

These additional fatty substances may be present in an amount of 0.1 to 50% by weight, especially 3 to 40% by weight and better still 5 to 30% by weight, relative to the total weight of the composition under consideration.

Surfactants

The first and/or second composition(s) of the product according to the invention may optionally comprise one or more surfactants, especially when they are in the form of an emulsion, in particular in an amount of from 0.01% to 30% by weight relative to the total weight of the composition containing it or them.

Mention may be made, alone or as a mixture, of alkali metal salts, ammonium salts, amine salts or amino alcohol salts of the following compounds: alkyl sulphates, alkyl ether sulphates, alkylamide sulphates and ether sulphates, alkylarylpolyether sulphates, monoglyceride sulphates, alkyl sulphonates, alkylamide sulphonates, alkylarylsulphonates, α-olefin sulphonates, paraffin sulphonates, alkyl sulphosuccinates, alkyl ether sulphosuccinates, alkylamide sulphosuccinates, alkyl sulphosuccinamates, alkyl sulphoacetates, alkylpolyglycerol carboxylates, alkyl phosphates/alkyl ether phosphates, acyl sarcosinates, alkyl polypeptidates, alkylamidopolypeptidates, acyl isethionates, alkyl laurates. The alkyl or acyl radical of all these compounds generally denotes a chain of 12 to 18 carbon atoms.

Mention may also be made of soaps and salts of fatty acids such as oleic acid, ricinoleic acid, palmitic acid, stearic acid and the acids of coconut oil or of hydrogenated coconut oil, and especially amine salts such as amine stearates; acyl lactylates in which the acyl radical contains 8-20 carbon atoms; polyglycol ether carboxylic acids.

Mention may also be made of polyethoxylated, polypropoxylated or polyglycerolated fatty alcohols, fatty alkylphenols and fatty acids, with a fatty chain containing from 8 to 18 carbon atoms; copolymers of ethylene oxide and of propylene oxide, condensates of ethylene oxide and of propylene oxide on fatty alcohols, polyethoxylated fatty amides, polyethoxylated fatty amines, ethanolamides, fatty acid esters of glycol, oxyethylenated or non-oxyethylenated fatty acid esters (stearate or oleate) of sorbitan, fatty acid esters of sucrose, fatty acid esters of polyethylene glycol (polyethylene glycol monostearate or monolaurate); phosphoric triesters, fatty acid esters of glucose derivatives; alkylpolyglycosides and alkylamides of amino sugars; the products of condensation of an α-diol, of a monoalcohol, of an alkylphenol, of an amide or of a diglycolamide with glycidol or a glycidol precursor.

Mention may also be made of trioleyl phosphate; fatty acid esters of polyols, for instance glyceryl or sorbitol mono-, di-, tri- or sesquioleates or stearates, and glyceryl or polyethylene glycol laurates; alkyl or alkoxy dimethicone copolyols with an alkyl or alkoxy chain that is pendent or at the end of the silicone skeleton, for example containing from 6 to 22 carbon atoms; polyoxyethylenated alkyl (lauryl, cetyl, stearyl or octyl) ethers and dimethicone copolyols.

Thickener

The first and/or second composition(s) of the product according to the invention may also comprise one or more thickeners, for example in concentrations of from 0.01% to 6% by weight relative to the total weight of the composition containing it or them.

The thickener may be chosen, alone or as a mixture, from:

polysaccharide biopolymers, for instance xanthan gum, carob gum, guar gum, alginates, modified celluloses such as hydroxyethylcellulose, methylcellulose, hydroxypropylcellulose and carboxymethylcellulose, starch derivatives, cellulose ether derivatives containing quaternary ammonium groups, and cationic polysaccharides;

synthetic polymers, for instance polyacrylic acids such as polyglyceryl (meth)acrylate polymers such as Hispagel or Lubragel from the companies Hispano Quimica or Gardian, polyvinylpyrrolidone, polyvinyl alcohol, crosslinked polymers of acrylamide and of ammonium acrylate, such as PAS 5161 or Bozepol C from Hoechst; acrylate/octylacrylamide copolymers, such as Dermacryl from National Starch; polyacrylamide-based polymers, such as Sepigel 305 from SEPPIC, crosslinked polymers of acrylamide and of methacryloyloxyethyltrimethylammonium chloride, such as Salcare SC 92 from Allied Colloids,

magnesium aluminium silicate;

optionally modified clays, for instance hectorites modified with a C₁₀ to C₂₂ fatty acid ammonium chloride, for instance hectorite modified with distearyldimethyl-ammonium chloride;

optionally modified silica;

galactomannans comprising one to six and better still from two to four hydroxyl groups per saccharide, substituted with a saturated or unsaturated alkyl chain, for instance guar gum alkylated with C₁ to C₆ and better still C₁ to C₃ alkyl chains and more particularly ethylated guar with a degree of substitution of 2 to 3, such as the product sold by the company Aqualon under the name N-Hance-AG;

cellulose derivatives such as ethylcellulose;

block copolymers, especially of “diblock” or “triblock” type, such as polystyrene/polyisoprene, polystyrene/polybutadiene, polystyrene/copoly(ethylene-butylene) or polystyrene/copoly(ethylene-propylene) such as those sold under the name “Kraton” by Shell Chemical;

polymers of polyamide type, for example comprising a polymer skeleton containing amide repeating units, and optionally at least one pendent fatty chain and/or at least one terminal chain, which are optionally functionalized, containing from 8 to 120 carbon atoms and being linked to these amide units, among which mention may be made of the products sold by the company Arizona Chemical under the names Uniclear 80 and Uniclear 100, which are a mixture of copolymers of a C₃₆ diacid condensed with ethylenediamine, with a weight-average molecular mass of about 6 000, and terminal ester groups resulting from the esterification of the remaining acid end groups with cetyl alcohol or stearyl alcohol, or mixtures thereof (also known as cetylstearyl alcohol).

Film-Forming Polymer

The first and/or second composition(s) of the product according to the invention may also comprise at least one film-forming polymer. This is generally the case when it is desired to prepare a product such as a nail varnish or a lip gloss.

Among the film-forming polymers that may be used in the product of the present invention, mention may be made of synthetic polymers, of free-radical type or of polycondensate type, and polymers of natural origin, and mixtures thereof.

The film-forming polymers of free-radical type may especially be vinyl polymers or copolymers, especially acrylic polymers.

The vinyl film-forming polymers may result from the polymerization of ethylenically unsaturated monomers containing at least one acid group and/or esters of these acidic monomers and/or amides of these acidic monomers, for instance α,β-ethylenic unsaturated carboxylic acids such as acrylic acid, methacrylic acid, crotonic acid, maleic acid or itaconic acid.

The vinyl film-forming polymers may also result from the homopolymerization or copolymerization of monomers selected from the group consisting of vinyl esters, for instance vinyl acetate, vinyl neodecanoate, vinyl pivalate, vinyl benzoate and vinyl t-butylbenzoate and styrene monomers, for instance styrene and α-methylstyrene. In particular, these monomers may be polymerized with acidic monomers and/or esters thereof and/or amides thereof, such as those mentioned above.

Among the film-forming polycondensates that may be mentioned are polyurethanes, polyesters, polyesteramides, polyamides, epoxy ester resins and polyureas.

The polymers of natural origin, which are optionally modified, may be selected from the group consisting of shellac resin, sandarac gum, dammar resins, elemi gums, copal resins, water-insoluble cellulose-based polymers such as nitrocellulose, cellulose acetate, cellulose acetobutyrate, cellulose acetopropionate or ethylcellulose, and mixtures thereof.

The polymers may be dissolved or dispersed in the cosmetically acceptable medium of the first and/or second composition. The polymers may be present in a content ranging from 0.01% to 40% by weight relative to the total weight of the composition containing it or them.

The first and/or second composition(s) of the product according to the invention may also comprise a plasticizer selected from the group consisting of the usual plasticizers, which may be present in a content ranging from 0.1% to 40% by weight relative to the total weight of the composition containing it.

Filler

The first and/or second composition(s) of the product according to the invention may also comprise one or more fillers compatible with cosmetic compositions.

The term “fillers” should be understood as meaning colorless or white, mineral or synthetic, lamellar or non-lamellar particles intended to give the composition body or rigidity, and/or give the makeup softness, a matt effect and uniformity.

The fillers, which may be present for example in a proportion of from 0.01% to 60% by weight and preferably 3% to 10%, in the first and/or second composition(s) of the product according to the invention, may be mineral or synthetic, and lamellar or non-lamellar. Mention may be made of talc, mica, silica, kaolin, Nylon powder, polyethylene powder, Teflon, starch, boron nitride, polymer microspheres such as Expancel (Nobel Industrie), Polytrap (Dow Corning) and silicone resin microbeads (for example Tospearls from Toshiba), precipitated calcium carbonate, magnesium carbonate, magnesium hydrocarbonate and metal soaps derived from organic carboxylic acids containing from 8 to 22 carbon atoms.

Additives

The first and second composition(s) of the makeup product according to the invention may also contain one or more cosmetic, dermatological, hygiene or pharmaceutical active agents.

As cosmetic, dermatological, hygiene or pharmaceutical active agents that may be used in the compositions of the invention, mention may be made of moisturizers (polyols, for instance glycerol), vitamins (C, A, E, F, B or PP), essential fatty acids, essential oils, ceramides, sphingolipids, liposoluble sunscreens or sunscreens in the form of nanoparticles, and specific active agents for treating the skin (protective agents, antibacterial agents, anti-wrinkle agents, etc.).

The product according to the invention may also be free of UV-screening agent.

These active agents are used in a usual amount for those skilled in the art and especially in concentrations of from 0 to 20% and especially from 0.001% to 15% of the total weight of the first or second composition.

Each composition of the product according to the invention may furthermore comprise, depending on the type of application envisaged, constituents conventionally used in the fields under consideration, which are present in an amount that is suitable for the desired presentation form.

Each composition of the product according to the invention may be in any form including any presentation form normally used for topical application, and especially in the form of an oily or aqueous solution, an oily or aqueous gel, an oil-in-water or water-in-oil emulsion, a multiple emulsion, a dispersion of oil in water by means of vesicles, the vesicles being located at the oil/water interface, or a powder. Each composition may be fluid or solid.

The compositions of the product according to the invention may be prepared in a manner that is usual for those skilled in the art.

They may be in the form of a cast product, for example in the form of a stick or tube, in the form of a soft paste in a hot-water bottle, or in the form of a cup that may be used by direct contact or with a sponge. In particular, they constitute, together or separately, a cast foundation, a cast makeup rouge or eyeshadow, which is especially colored, a lipstick, a lip gloss, a concealer product or an eye contour product.

They may also each be in the form of a soft paste, an ointment, a salve, a lotion, a gel or a more or less fluid cream. In this case, they may constitute fluid or pasty foundations or lipsticks, lip glosses, antisun products, skin-coloring products, eyeliners or body makeup products, or alternatively may have care properties and may then be in the form of a lipcare base or balm.

Advantageously, the first or second composition, or both, contain(s) a continuous fatty phase and is(are) preferably in anhydrous form and may contain less than 5% water and better still less than 1% water relative to the total weight of each of the compositions. In particular, the entire two-coat makeup product is in anhydrous form.

Preferably, each composition is in the form of a more or less rigid stick.

Each composition may be packaged separately in the same packaging article, for example in a two-compartment pen.

Preferably, the composition that is applied as the first coat is in solid form, which allows a more practical application, better stability of the composition over time and to temperature, and allows accurate marking of the makeup, which is highly desirable in the case of a lipstick or an eyeliner.

The product according to the invention may be advantageously used for making up the skin, the lips and/or the integuments depending on the nature of the ingredients used.

Preferably, the product according to the invention is a skin or lip makeup product. More particularly, it is in the form of a two-coat lipstick.

### EXAMPLES

The percentages below are all expressed on a weight basis relative to the total weight of the composition.

The three photochromic dyes used in the examples below are naphthopyran derivatives from the company James Robinson sold under the following names:

| | | |
|---|---|---|
| Reversacol | 195 | Ref. 1 |
| Reversacol | 208 | Ref. 2 |
| Reversacol | 306 | Ref. 3 |

Their respective ΔE values were assessed according to the protocol described in the description. Table I gives the differences in hue (ΔE) obtained after exposure for 2 minutes.

| Photochromic agents | ΔE |
|---|---|
| Ref. 1 | 39 |
| Ref. 2 | 6 |
| Ref. 3 | 34 |

Each of these three photochromic dyes is formulated in a lipstick base having the composition as below and intended to feature one of the two components of a two-coat lipstick.

Base lipstick formulation:

| | |
|---|---|
| 2-octyldodecanol | 0.5% |
| hectorite modified with distearyl dimethyl- | 0.6% |
| ammonium chloride | |
| liquid lanolin | 27.2% |
| microcrystalline wax | 10.5% |
| polyglycerolated (3 mol) beeswax | 4.2% |
| acetylated lanolin | 6.7% |
| arara oil (oleic acid esters) | 13.5% |
| oxypropylenated (5 PO) lanolin wax | 6.7% |
| oleyl erucate | 13.5% |
| oleic-linoleic-linolenic acid | 1.7% |
| triglycerides | |
| palmitic-oleic-linoleic acid triglycerides | 13.5% |
| sodium hyaluronate | 0.1% |
| preserving agents | 0.1% |
| vitamin | 0.5% |
| UV-screening agents | 0.7% |
| pigments | 7% |
| photochromic dye under consideration | 2% |

For each of the three formulations thus obtained, a gloss formulation coat comprising 95% polybutylene and 5% Helicone sold by the company Wacker is superimposed on a coat thereof.

On exposure to sunlight, the applied product changes color significantly for each of the three products tested. The exposure to sunlight reveals the Helicones. This color change is particularly pronounced with the formulations comprising the dyes Ref. 1 and 3, respectively.

The above written description of the invention provides a manner and process of making and using it such that any person skilled in this art is enabled to make and use the same, this enablement being provided in particular for the subject matter of the appended claims, which make up a part of the original description.

All references, patents, applications, tests, standards, documents, publications, brochures, texts, articles, etc. mentioned herein are incorporated herein by reference. Where a numerical limit or range is stated, all values and subranges therewithin are specifically included as if explicitly written out.

As used above, the phrase “selected from the group consisting of” includes mixtures of the specified materials.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A cosmetic product comprising at least a first composition and a second composition, the first composition comprising at least one first dye in a physiologically acceptable medium, the second composition comprising at least one second dye in a physiologically acceptable medium, the first dye being photochromic and the second dye being at least one goniochromatic coloring agent.

2. The product according to claim 1, wherein the photochromic dye is organic.

3. The product according to claim 2, wherein the photochromic dye is a naphthopyran.

4. The product according to claim 1, wherein at least one of the first and second compositions comprise a fatty phase and an aqueous phase.

5. The product according to claim 1, wherein the first dye is at least one 2H-naphtho[2,1-b]pyran of formula (I) or a 3H-naphtho[2,1-b]pyran of formula (II): 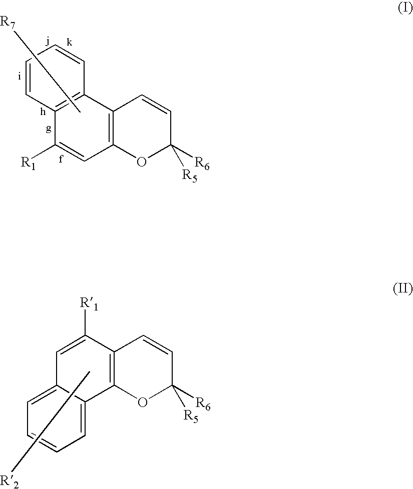
in which:
R₁ represents:
(i) a hydrogen atom;
(ii) a linear, branched or cyclic, saturated or unsaturated hydrocarbon-based group containing from 1 to 30 carbon atoms, optionally comprising 1 to 5 hetero atoms selected from the group consisting of N, O, S, Si and P, and/or optionally halogenated or perhalogenated;
(iii) a hydrocarbon-based ring formed with one of the bonds “f” or “gh” and the radical R₇; or
(iv) a group selected from the group consisting of —COOR₄, —C(O)NR₂R₃, —NR₂R₃, —OR₄ and —SR₄, in which:
R₂ and R₃ either represent, independently of each other, a linear, branched or cyclic, saturated or unsaturated hydrocarbon-based group containing 1 to 20 carbon atoms, optionally comprising 1 to 5 hetero atoms selected from the group consisting of N, O, S, Si and P,
or, taken together with the nitrogen atom to which they are attached, form a saturated or unsaturated hydrocarbon-based heterocycle containing 3 to 10 carbon atoms and optionally 1 to 5 other hetero atoms selected from the group consisting of N, O, S, Si and P, the ring optionally being substituted with at least one linear, branched or cyclic, saturated or unsaturated hydrocarbon-based radical containing 1 to 20 carbon atoms, optionally comprising 1 to 5 hetero atoms selected from the group consisting of N, O, S, Si and P;
R₄ represents a linear, branched or cyclic, saturated or unsaturated hydrocarbon-based group containing 1 to 20carbon atoms, which is optionally halogenated or perhalogenated, and/or optionally comprising 1 to 5 hetero atoms selected from the group consisting of N, O, S, Si and P;
R₅ and R₆ represent, independently of each other, a group selected from the group consisting of:
(i) the saturated cyclic aminoaryl groups of formula (IIA) or (IIB): 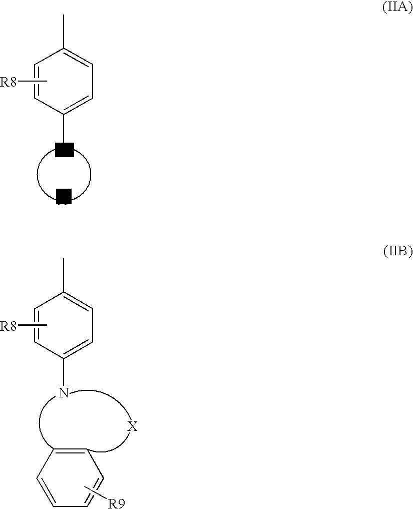
in which the ring comprising N and X is a saturated ring containing in total 3 to 30 atoms atoms, including the nitrogen, the remainder being carbon atoms and/or hetero atoms selected from the group consisting of O, S, Si and P and/or groups selected from the group consisting of —NH and —NR with R representing a linear, branched or cyclic, saturated or unsaturated hydrocarbon-based radical containing 1 to 20 carbon atoms, optionally comprising 1 to 5 hetero atoms selected from the group consisting of N, O, S, Si and P;
(ii) the indolinoaryl groups of formula (III): 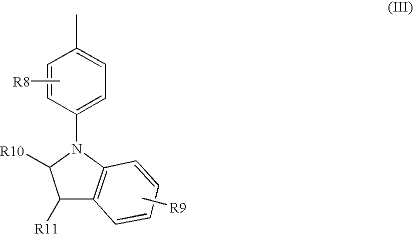
in which R₁₀ and R₁₁ represent, independently of each other, a group selected from the group consisting of (i) linear, branched or cyclic, saturated or unsaturated hydrocarbon-based groups containing 1 to 30 carbon atoms, optionally comprising 1 to 5 hetero atoms selected from the group consisting of N, O, S, Si and P, and/or optionally halogenated or perhalogenated; (ii) halogen atoms; (iii) —CN (nitrile), —COOH (carboxylate) or —NO₂ (nitro) groups; (iv) a hydrogen atom; (v) a group selected from the group consisting of —C(O)NR₂R₃, —NR₂R₃, —OR₄ and —SR₄ with R₂, R₃ and R₄ having the meanings given above; (vi) the radicals R₁₀ and R₁₁ together possibly forming a saturated or unsaturated hydrocarbon-based ring containing in total 5 to 8 atoms (including the atoms of the indoline ring), the atoms being selected from the group consisting of C, O, S and/or NR with R representing H or a linear, branched or cyclic, saturated or unsaturated hydrocarbon-based radical containing 1 to 20 carbon atoms, optionally comprising 1 to 5 hetero atoms selected from the group consisting of N, O, S, Si and P,
(iii) the groups of formula (IV): 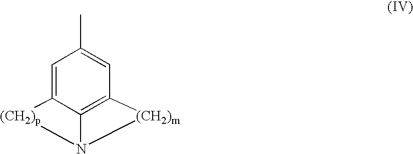
in which m and p are, independently of each other, integers ranging from 2 to 5;
(iv) the unsaturated cyclic aminoaryl groups of formula (VA), (VB) or (VC): 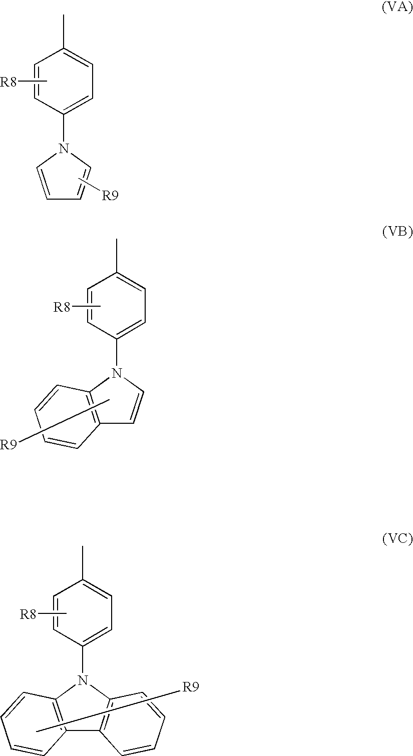
in which R₈ and R₉ represent, independently of each other, a group selected from the group consisting of (i) linear, branched or cyclic, saturated or unsaturated hydrocarbon-based groups containing 1 to 30 carbon atoms, optionally comprising 1 to 5 hetero atoms selected from the group consisting of N, O, S, Si and P, and/or optionally halogenated or perhalogenated,; (ii) halogen atoms; (iii) —CN (nitrile), —COOH (carboxylate) or —NO₂ (nitro) groups; (iv) a hydrogen atom; (v) a group selected from the group consisting of —C(O)NR₂R₃, —NR₂R₃, —OR₄ and —SR₄ with R₂, R₃ and R₄ having the meanings given above;
(v) a linear, branched or cyclic, saturated or unsaturated hydrocarbon-based group containing 1 to 30 carbon atoms, optionally comprising 1 to 5 hetero atoms selected from the group consisting of N, O, S, Si and P; and especially a group selected from the group consisting of —C₆H₄—CONR₂R₃, —C₆H₄—NR₂R₃ and —C₆H₄—OR₄ with R₂, R₃ and R₄ having the meanings given above;
R₇ represents a group selected from the group consisting of:
(i) linear, branched or cyclic, saturated or unsaturated hydrocarbon-based groups containing 1 to 30 carbon atoms, optionally comprising 1 to 5 hetero atoms selected from the group consisting of N, O, S, Si and P, and/or optionally halogenated or perhalogenated;
(ii) halogen atoms;
(iii) —CN (nitrile), —COOH (carboxylate), —NO₂ (nitro), —N═N— (azo), ═NH (imino) or —CONH₂ (amide) groups;
(iv) a hydrogen atom;
(v) a group selected from the group consisting of —C(O)NR₂R₃, —NR₂R₃, —OR₄ and —SR₄ with R₂, R₃ and R₄ having the meanings given above;
(vi) the radical R₇ also possibly forming, with one of the bonds “i”, “j”, “k” or “g,h” taken with the radical R₁, or “f” taken with the radical R₁, a saturated hydrocarbon-based ring containing in total 3 to 8 carbon atoms, optionally comprising 1 to 5 hetero atoms selected from the group consisting of N, O, S, Si and P;
R′₁ represents a group selected from the group consisting of:
(i) a hydrogen atom;
(ii) a linear, branched or cyclic, saturated or unsaturated hydrocarbon-based group containing 1 to 30 carbon atoms, optionally comprising 1 to 5 hetero atoms selected from the group consisting of N, O, S, Si and P, and/or optionally halogenated or perhalogenated;
(iii) a group selected from the group consisting of —C(O)NR₂R₃, —NR₂R₃, —OR₄ and —SR₄, with R₂, R₃ and R₄ having the meanings given above;
R′₂ represents a group selected from the group consisting of:
(i) linear, branched or cyclic, saturated or unsaturated hydrocarbon-based groups containing 1 to 30 carbon atoms, optionally comprising 1 to 5 hetero atoms selected from the group consisting of N, O, S, Si and P, and/or optionally halogenated or perhalogenated;
(ii) halogen atoms;
(iii) —CN (nitrile), —COOH (carboxylate), —NO₂ (nitro), —N═N— (azo), ═NH (imino) or —CONH₂ (amide) groups;
(iv) a hydrogen atom;
(v) a group selected from the group consisting of —C(O)NR₂R₃, —NR₂R₃, —OR₄ and —SR₄, with R₂, R₃ and R₄ having the meanings given above.

6. The product according to claim 5, comprising a first dye corresponding to formula (Ia) or (IIa) below: 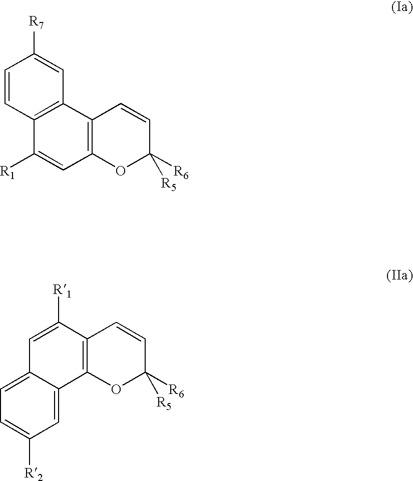
in which R₁, R₅, R₆, R₇, R′₁ and R′₂ are as defined in claim 5.

7. The product according to claim 5, wherein R₁ represents a hydrogen atom; a hydrocarbon-based ring with one of the bonds “f” or “gh” and the radical R₇; or a group selected from the group consisting of —COOR₄, —NR₂R₃, —OR₄ and —SR₄, in which:
R₂ and R₃ either may represent, independently of each other, a linear, branched or cyclic, saturated or unsaturated hydrocarbon-based group containing 1 to 20 carbon atoms, optionally comprising 1 to 5 hetero atoms selected from the group consisting of N, O, S, Si and P,
or, taken together with the nitrogen atom to which they are attached, may form a saturated or unsaturated hydrocarbon-based heterocycle containing 3 to 10 carbon atoms and optionally 1 to 5 other hetero atoms selected from the group consisting of N, O, S, Si and P, the ring optionally being substituted with at least one linear, branched or cyclic, saturated or unsaturated hydrocarbon-based radical containing 1 to 20 carbon atoms, optionally comprising 1 to 5 hetero atoms selected from the group consisting of N, O, S, Si and P;
and/or
R₄ represents a linear, branched or cyclic, saturated or unsaturated hydrocarbon-based group containing 1 to 20 carbon atoms, optionally halogenated or perhalogenated and/or optionally comprising 1 to 5 hetero atoms selected from the group consisting of N, O, S, Si and P;
and/or
R₅ and R₆ may represent, independently of each other, a group selected from the group consisting of:
the saturated cyclic aminoaryl groups of formula (IIA) or (IIB): 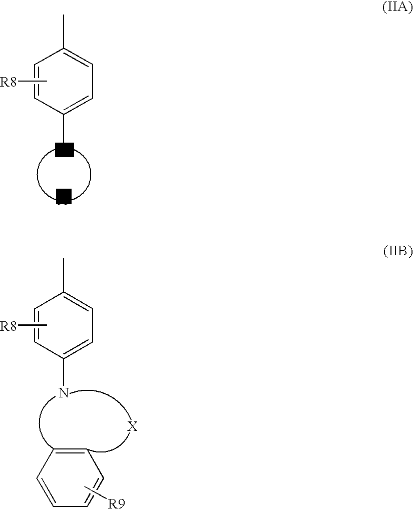
in which the ring comprising N and X is a saturated ring which contains in total 3 to 30 atoms, including nitrogen, the rest being carbon atoms and/or hetero atoms selected from the group consisting of O, S, Si and P and/or groups selected from the group consisting of —NH and —NR with R representing a linear, branched or cyclic, saturated or unsaturated hydrocarbon-based radical containing 1 to 20 carbon atoms, optionally comprising 1 to 5 hetero atoms selected from the group consisting of N, O, S, Si and P;
a linear, branched or cyclic, saturated or unsaturated hydrocarbon-based group containing 1 to 30 carbon atoms, optionally comprising 1 to 5 hetero atoms selected from the group consisting of N, O, S, Si and P; and especially a group selected from the group consisting of —C₆H₄—CONR₂R₃, —C₆H₄—NR₂R₃ and —C₆H₄—OR₄ with R₂, R₃ and R₄ having the meanings given above.

8. The product according to claim 5, wherein the first dye corresponds to formula (I) for which:
R₁ represents hydrogen; or a group —COOR with R being a saturated hydrocarbon-based radical containing 1 to 12 carbon atoms,; or a group 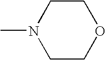
and/or
R₅ and R₆ represent, independently of each other, either (i) a group of formula (IIA): 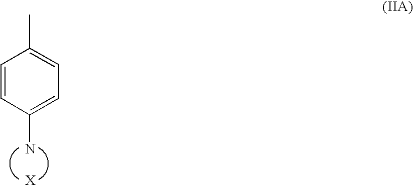
in which the ring comprising N and X is a saturated ring containing in total 4 to 7 atoms , including nitrogen, and especially 3 to 5 carbon atoms and 0 or 1 oxygen atom; and in particular a group of formula: 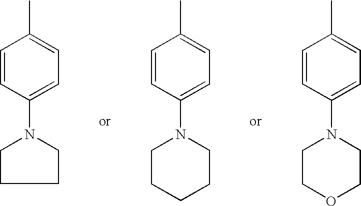
or (ii) a linear, branched or cyclic, saturated or unsaturated hydrocarbon-based group containing 5 to 14 carbon atoms, optionally comprising 1 or 2 hetero atoms selected from the group consisting of N, O or S;
and/or
R₇ represents a hydrogen atom or a group —NR₂R₃, with R₂ and R₃ representing, independently of each other, a linear or branched, saturated hydrocarbon-based group containing 1 to 12 carbon atoms.

9. The product according to claim 5, wherein the first dye corresponds to formula (II) for which:
R′₁ represents hydrogen or a group —COOR with R being a saturated hydrocarbon-based radical containing 1 to 12;
and/or
R₅ and R₆ represent, independently of each other, either (i) a group of formula (IIA): 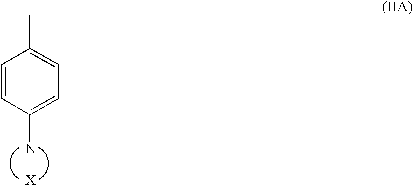
in which the ring comprising N and X is a saturated ring containing in total 4 to 7 atoms including nitrogen,
or (ii) a linear, branched or cyclic, saturated or unsaturated hydrocarbon-based group containing 5 to 14 carbon atoms, optionally comprising 1 or 2 hetero atoms selected from the group consisting of N, O and S;
and/or
R′₂ represents hydrogen or a group —NR′R″, with R′ and R″, which may be identical or different, representing a linear or branched, saturated hydrocarbon-based group containing 1 to 12 carbon atoms.

10. The product according to claim 1, wherein the first dye is selected from the group consisting of:
3,3-di(4-methoxyphenyl)-6-morpholino-3H-naphtho[2,1-b]pyran
3-phenyl-3-(4-morpholinophenyl)-6-morpholino-3H-naphtho[2,1-b]pyran
3-phenyl-3-(4-piperidinophenyl)-6-morpholino-3H-naphtho[2,1-b]pyran
3-phenyl-3-(4-piperidinophenyl)-6-carboxymethyl-9-N-dimethyl-3H-naphtho[2,1-b]pyran
2-phenyl-2-(4-piperidinophenyl)-5-carboxymethyl-9-N-dimethyl-2H-naphtho[1,2-b]pyran, and
mixtures thereof.

11. The product according to claim 1, wherein the first dye(s) is(are) present in a proportion of from 0.001% to 20% by weight, relative to the total weight of the composition containing it or them.

12. The product according to claim 1, comprising at least two different first dyes in the same composition.

13. The product according to claim 1, wherein the goniochromatic coloring agent is present in a proportion of from 0.1% to 60% by weight, relative to the total weight of the composition containing it.

14. The product according to claim 1, wherein the goniochromatic coloring agent is selected from the group consisting of multilayer interference structures and liquid-crystal coloring agents.

15. The product according to claim 14, comprising a goniochromatic coloring agent of multilayer interference structure comprising at least two layers, each layer, optionally independently of the other layer(s), being made from at least one material selected from the group consisting of the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys and polymers, and combinations thereof.

16. The product according to claim 14, comprising a liquid-crystal coloring agent obtained by polymerization of a monomer mixture comprising:
a) at least a first monomer A of formula (I) Y1-A1-M1-A2-Y2 in which
i) Y1 and Y2, which may be identical or different, represent an acrylate or methacrylate group;
ii) A1 and A2, which may be identical or different, represent a group of formula —CnH₂n—, in which n is an integer ranging from 1 to 20;
iii) M1 denotes a group of general formula (I′) —R₁—X₁—R₂—X₂—R₃—X₃—R₄—, in which R₁ and R₄ denote —O—, and R₂ and R₃ denote —COO—,
and X₁, X₂ and X₃ are a 1,4-phenylene group, the carbonyl group —CO— of R₂ and of R₃, respectively, being linked to the group X₁ or X₃, respectively,
and
b) at least a second monomer B, which is chiral, of formula (II) V1-W1-Z-W2-V2, in which
i) V1 denotes an acrylate or methacrylate group, and V2 denotes a C₁-C₂₀ alkyl, C₁-C₂₀ alkoxy, (C₁-C₂₀)alkoxycarbonyl or —OH group;
ii) W1 represents a divalent group of formula —X′1—CO—O—,
W2 represents a divalent group of formula —O—CO—X′1—,
in which X′1 denotes a 1,4-phenylene group,
and Z denotes a chiral group containing two bonds, derived from the dianhydrohexite group, in particular a divalent radical of formula:

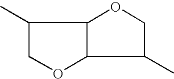

17. The product according to claim 16, wherein the liquid-crystal coloring agent is obtained from a monomer mixture comprising from 70% to 99% by weight of monomer A and from 1% to 30% by weight of monomer B, relative to the total weight of monomer A and of monomer B.

18. The product according to claim 14, wherein the liquid-crystal coloring agent is present in a proportion of from 0.01% to 99% by weight, relative to the total weight of the composition containing it.

19. The product according to claim 1, wherein the first and/or second composition(s) further comprise at least one non-goniochromatic dye that is different from the first dye.

20. The product according to claim 19, wherein the dye is selected from the group consisting of water-soluble or liposoluble monochromatic dyes, pigments, reflective particles and nacres, and mixtures thereof.

21. The product according to claim 1, wherein the first and/or second composition(s) further comprise(s) at least one liposoluble or water-soluble monochromatic dye.

22. The product according to claim 1, wherein the first and/or second composition(s) further comprise(s) at least one liposoluble or water-soluble monochromatic dye in a proportion of from 0.001% to 15% by weight relative to the total weight of the composition containing it.

23. The product according to claim 1, wherein the first and/or second composition(s) further comprise(s) at least one pigment.

24. The product according to claim 1, wherein the first and/or second composition(s) further comprise(s) at least one pigment in a proportion of from 0.01% to 25% by weight relative to the total weight of the composition containing it.

25. The product according to claim 1, wherein the first and/or second composition(s) further comprise(s) at least one nacre.

26. The product according to claim 1, wherein the first and/or second composition(s) further comprise(s) at least one nacre in a proportion of from 0.01% to 20% by weight relative to the total weight of the composition containing it.

27. The product according to claim 1, wherein the first and/or second composition(s) further comprise(s) at least reflective particles.

28. The product according to claim 1, wherein the first and/or second composition(s) further comprise(s) at last reflective particles in a proportion of from 0.1% to 20% by weight relative to the total weight of the composition containing them.

29. The product according to claim 1, wherein the first and/or second composition(s) comprise(s) at least one oily phase.

30. The product according to claim 29, wherein the oily phase comprises one or more polar or apolar, volatile or non-volatile oils.

31. The product according to claim 29, wherein the oily phase comprises from 5% to 100% by weight of polar oil(s) relative to the total weight of the oily phase.

32. The product according to claim 29, wherein the oily phase comprises from 5% to 100% by weight of a polar oil(s) relative to the total weight of the oily phase.

33. The product according to claim 29, wherein the oils are be selected from the group consisting of volatile or non-volatile oils of animal, plant, mineral or synthetic origin, and mixtures thereof.

34. The product according to claim 33, wherein the oils are selected from the group consisting of animal or plant oils, synthetic esters and ethers, especially of fatty acids, fatty alcohols, linear or branched hydrocarbons of mineral or synthetic origin, and glycerides, and mixtures thereof.

35. The product according to claim 29, wherein the oily phase is such that the first dye(s) is(are) soluble therein.

36. The product according to claim 29, wherein the oily phase is present in a proportion of from 1% to 99% by weight relative to the total weight of the composition containing it.

37. The product according to claim 1, wherein the first and/or second composition(s) further comprise(s) from 0.1% to 50% by weight of a fatty substance other than an oil, relative to the total weight of the composition containing it.

38. The product according to claim 1, wherein the first and/or second composition(s) is(are) anhydrous.

39. The product according to claim 1, wherein the first and/or second composition(s) comprise(s) at least one aqueous phase.

40. The product according to claim 39, wherein the aqueous phase comprises from 0.1% to 14% by weight of a C₂-C₆ monoalcohol, relative to the total weight of the aqueous phase.

41. The product according to claim 1, wherein the first and/or second composition(s) further comprise(s) at least one surfactant.

42. The product according according to claim 1, wherein the first and/or second composition(s) further comprise(s) at least one surfactant in an amount ranging from 0.01% to 30% by weight relative to the total weight of the composition containing it.

43. The product according to claim 1, wherein the first and/or second composition(s) further comprise(s) at least one thickener.

44. The product according to claim 1, wherein the first and/or second composition(s) further comprise(s) at least one thickener in a proportion of from 0.01% to 6% by weight relative to the total weight of the composition containing it.

45. The product according to claim 1, wherein the first and/or second composition(s) further comprise(s) at least one film-forming polymer.

46. The product according to claim 1, wherein the first and/or second composition(s) further comprise(s) at least one film-forming polymer in a proportion of from 0.01% to 40% by weight relative to the total weight of the composition containing it.

47. The product according to claim 1, wherein the first and/or second composition(s) further comprise(s) at least one filler.

48. The product according to claim 1, wherein wherein the first and/or second composition(s) further comprise(s) at least one filler in a proportion of from 0.01% to 60% by weight relative to the total weight of the composition containing it.

49. A process for making up the skin, the lips and/or the integuments, comprising the application of the product according to claim 1 to the skin, the lips and/or the integuments.

50. The process according to claim 49, wherein a first composition comprising at least the first dye in a physiologically acceptable medium is applied, as a first coat, and a coat of the second composition comprising at least one goniochromatic coloring agent in a physiologically acceptable medium is then applied over all or part of the first coat.

51. A makeup kit comprising a product according to claim 1.

52. The kit according to claim 51, further comprising means for applying the first and the second composition to the skin, the lips and/or the integuments.

53. The kit according to claim 51, further comprising application means selected from the group consisting of brushes, pens, pencils, felts, nibs, sponges and foams.

54. The kit according to claim 51, wherein the first and second compositions are packaged in separate packaging articles.

55. A cosmetic product comprising at least a first composition and a second composition, the first composition comprising at least one first dye in a physiologically acceptable medium, and the second composition comprising at least one second dye in a physiologically acceptable medium, the first dye being a naphthopyran and the second dye being at least one goniochromatic coloring agent.
